SEQUENCE LISTING (54 sequences)

SEQ ID NO: 1 - DNA, 40 bp, Homo sapiens
gctgctactcgcctgcccaggtgggcaggggctggagagg

SEQ ID NO: 2 - DNA, 40 bp, Homo sapiens
ccccgtccccgtccgcctaggagaacctgtggctggacag

SEQ ID NO: 3 - DNA, 5562 bp, Homo sapiens
atggctgagagcgcctccccgccctcctcatctgcagcagccccagccgctgagccaggagtcaccacggagcagcccggaccccggagccccccatcttccccgccaggcctggaggagcctctggatggagctgatcctcatgtcccacacccagacctggcgcctattgccttcttctgcctgcgacagaccaccagcccccggaactggtgcatcaagatggtgtgcaacccgtggtttgaatgtgtcagcatgctggtgatcctgctgaactgcgtgacacttggcatgtaccagccgtgcgacgacatggactgcctgtccgaccgctgcaagatcctgcaggtctttgatgacttcatctttatcttctttgccatggagatggtgctcaagatggtggccctggggatttttggcaagaagtgctacctcggggacacatggaaccgcctggatttcttcatcgtcatggcagggatggtcgagtactccctggaccttcagaacatcaacctgtcagccatccgcaccgtgcgcgtcctgaggcccctcaaagccatcaaccgcgtgcccagtatgcggatcctggtgaacctgctcctggacacactgcccatgctggggaatgtcctgctgctctgcttctttgtcttcttcatctttggcatcataggtgtgcagctctgggcgggcctgctgcgtaaccgctgcttcctggaggagaacttcaccatacaaggggatgtggccttgcccccatactaccagccggaggaggatgatgagatgcccttcatctgctccctgtcgggcgacaatgggataatgggctgccatgagatccccccgctcaaggagcagggccgtgagtgctgcctgtccaaggacgacgtctacgactttggggcggggcgccaggacctcaatgccagcggcctctgtgtcaactggaaccgttactacaatgtgtgccgcacgggcagcgccaacccccacaagggtgccatcaactttgacaacatcggttatgcttggattgtcatcttccaggtgatcactctggaaggctgggtggagatcatgtactacgtgatggatgctcactccttctacaacttcatctacttcatcctgcttatcatagtgggctccttcttcatgatcaacctgtgcctcgttgtcatagcgacccagttctcggagaccaagcaacgggagcaccggctgatgctggagcagcggcagcgctacctgtcctccagcacggtggccagctacgccgagcctggcgactgctacgaggagatcttccagtatgtctgccacatcctgcgcaaggccaagcgccgcgccctgggcctctaccaggccctgcagagccggcgccaggccctgggcccggaggccccggcccccgccaaacctgggccccacgccaaggagccccggcactaccagctgtgcccgcaacatagccccctggatgcgacgccccacaccctggtgcagcccatccccgccacgctggcttccgatcccgccagctgcccttgctgccagcatgaggacggccggcggccctcgggcctgggcagcaccgactcgggccaggagggctcgggctccgggagctccgctggtggcgaggacgaggcggatggggacggggcccggagcagcgaggacggagcctcctcagaactggggaaggaggaggaggaggaggagcaggcggatggggcggtctggctgtgcggggatgtgtggcgggagacgcgagccaagctgcgcggcatcgtggacagcaagtacttcaaccggggcatcatgatggccatcctggtcaacaccgtcagcatgggcatcgagcaccacgagcagccggaggagctgaccaacatcctggagatctgcaatgtggtcttcaccagcatgtttgccctggagatgatcctgaagctggctgcatttgggctcttcgactacctgcgtaacccctacaacatcttcgacagcatcattgtcatcatcagcatctgggagatcgtggggcaggcggacggtgggctgtcggtgctgcggaccttccggctgctgcgcgtgctgaaactggtgcgcttcatgcctgccctgcggcgccagctcgtggtgctcatgaagaccatggacaacgtggccaccttctgcatgctgctcatgctcttcatcttcatcttcagcatccttgggatgcatatttttggctgcaagttcagcctccgcacggacactggagacacggtgcccgacaggaagaacttcgactccctgctgtgggccatcgtcactgtgttccagatcctcacccaggaggactggaacgtcgttctctacaatggcatggcctccacttctccctgggcctccctctactttgtcgccctcatgaccttcggcaactatgtgctcttcaacctgctggtggccatcctggtggagggcttccaggcggagggtgacgccaatcgctcctactcggacgaggaccagagctcatccaacatagaagagtttgataagctccaggaaggcctggacagcagcggagatcccaagctctgcccaatccccatgacccccaatgggcacctggaccccagtctcccactgggtgggcacctaggtcctgctggggctgcgggacctgccccccgactctcactgcagccggaccccatgctggtggccctgggctcccgaaagagcagcgtcatgtctctagggaggatgagctatgaccagcgctccctgtccagctcccggagctcctactacgggccatggggccgcagcgcggcctgggccagccgtcgctccagctggaacagcctcaagcacaagccgccgtcggcggagcatgagtccctgctctctgcggagcgcggcggcggcgcccgggtctgcgaggttgccgcggacgaggggccgccgcgggccgcacccctgcacaccccacacgcccaccacgttcatcacgggccccatctggcgcaccgccaccgccaccaccgccggacgctgtccctcgacaacagggactcggtggacctggccgagctggtgcccgcggtgggcgcccacccccgggccgcctggagggcggcaggcccggcccccgggcatgaggactgcaatggcaggatgcccagcatcgccaaagacgtcttcaccaagatgggcgaccgcggggatcgcggggaggatgaggaggaaatcgactacaccctgtgcttccgcgtccgcaagatgatcgacgtctataagcccgactggtgcgaggtccgcgaagactggtctgtctacctcttctctcccgagaacaggttccgggtcctgtgtcagaccattattgcccacaaactcttcgactacgtcgtcctggccttcatctttctcaactgcatcaccatcgccctggagcggcctcagatcgaggccggcagcaccgaacgcatctttctcaccgtgtccaactacatcttcacggccatcttcgtgggcgagatgacattgaaggtagtctcgctgggcctgtacttcggcgagcaggcgtacctacgcagcagctggaacgtgctggatggctttcttgtcttcgtgtccatcatcgacatcgtggtgtccctggcctcagccgggggagccaagatcttgggggtcctccgagtcttgcggctcctgcgcaccctacgccccctgcgtgtcatcagccgggcgccgggcctgaagctggtggtggagacactcatctcctccctcaagcccatcggcaacatcgtgctcatctgctgtgccttcttcatcatctttggcatcctgggagtgcagctcttcaagggcaagttctaccactgtctgggcgtggacacccgcaacatcaccaaccgctcggactgcatggccgccaactaccgctgggtccatcacaaatacaacttcgacaacctgggccaggctctgatgtccctctttgtcctggcatccaaggatggttgggtgaacatcatgtacaatggactggatgctgttgctgtggaccagcagcctgtgaccaaccacaacccctggatgctgctgtacttcatctccttcctgctcatcgtcagcttctttgtgctcaacatgtttgtgggtgtcgtggtggagaacttccacaagtgccggcagcaccaggaggctgaagaggcacggcggcgtgaggagaagcggctgcggcgcctggagaagaagcgccggaaggcccagcggctgccctactatgccacctattgtcacacccggctgctcatccactccatgtgcaccagccactacctggacatcttcatcaccttcatcatctgcctcaacgtggtcaccatgtccctggagcactacaatcagcccacgtccctggagacagccctcaagtactgcaactatatgttcaccactgtctttgtgctggaggctgtgctgaagctggtggcatttggtctgaggcgcttcttcaaggaccgatggaaccagctggacctggccattgtgctactgtcagtcatgggcatcaccctggaggagatcgagatcaatgcggccctgcccatcaatcccaccatcatccgcatcatgagggttctgcgcattgcccgagtgctgaagctgttgaagatggccacaggaatgcgggccctgctggacacggtggtgcaagctttgccccaggtgggcaacctgggcctcctcttcatgctgctcttcttcatctatgctgctctcggggtggagctctttgggaagctggtctgcaacgacgagaacccgtgcgagggcatgagccggcatgccaccttcgagaacttcggcatggccttcctcacactcttccaggtctccacgggtgacaactggaacgggatcatgaaggacacgctgcgggactgcacccacgacgagcgcagctgcctgagcagcctgcagtttgtgtcgccgctgtacttcgtgagcttcgtgctcaccgcgcagttcgtgctcatcaacgtggtggtggctgtgctcatgaagcacctggacgacagcaacaaggaggcgcaggaggacgccgagatggatgccgagctcgagctggagatggcccatggcctgggccctggcccgaggctgcctaccggctccccgggcgcccctggccgagggccgggaggggcgggcggcgggggcgacaccgagggcggcttgtgccggcgctgctactcgcctgcccaggtgggcaggggctggagaggtgcgagggtcgccagagggggggcaccgcagggccagatgggcagcagggccagggaggccttgcacagagacctctgtctttctgggccccaccctgctcagctccatagaaggggtgaccctgtgatgagggggtcggtcccaggccatgggcgcagagaaccaaccgggagggcagctgtgtcctgcccaccccacagccccgggacccggctgataatcccgcctgtccccaccccgtccccgtccgcc

SEQ ID NO: 4 - protein, 1854 aa, Homo sapiens
MAESASPPSSSAAAPAAEPGVTTEQPGPRSPPSSPPGLEEPLDGADPHVPHPDLAPIAFFCLRQTTSPRNWCIKMVCNPWFECVSMLVILLNCVTLGMYQPCDDMDCLSDRCKILQVFDDFIFIFFAMEMVLKMVALGIFGKKCYLGDTWNRLDFFIVMAGMVEYSLDLQNINLSAIRTVRVLRPLKAINRVPSMRILVNLLLDTLPMLGNVLLLCFFVFFIFGIIGVQLWAGLLRNRCFLEENFTIQGDVALPPYYQPEEDDEMPFICSLSGDNGIMGCHEIPPLKEQGRECCLSKDDVYDFGAGRQDLNASGLCVNWNRYYNVCRTGSANPHKGAINFDNIGYAWIVIFQVITLEGWVEIMYYVMDAHSFYNFIYFILLIIVGSFFMINLCLVVIATQFSETKQREHRLMLEQRQRYLSSSTVASYAEPGDCYEEIFQYVCHILRKAKRRALGLYQALQSRRQALGPEAPAPAKPGPHAKEPRHYQLCPQHSPLDATPHTLVQPIPATLASDPASCPCCQHEDGRRPSGLGSTDSGQEGSGSGSSAGGEDEADGDGARSSEDGASSELGKEEEEEEQADGAVWLCGDVWRETRAKLRGIVDSKYFNRGIMMAILVNTVSMGIEHHEQPEELTNILEICNVVFTSMFALEMILKLAAFGLFDYLRNPYNIFDSIIVIISIWEIVGQADGGLSVLRTFRLLRVLKLVRFMPALRRQLVVLMKTMDNVATFCMLLMLFIFIFSILGMHIFGCKFSLRTDTGDTVPDRKNFDSLLWAIVTVFQILTQEDWNVVLYNGMASTSPWASLYFVALMTFGNYVLFNLLVAILVEGFQAEGDANRSYSDEDQSSSNIEEFDKLQEGLDSSGDPKLCPIPMTPNGHLDPSLPLGGHLGPAGAAGPAPRLSLQPDPMLVALGSRKSSVMSLGRMSYDQRSLSSSRSSYYGPWGRSAAWASRRSSWNSLKHKPPSAEHESLLSAERGGGARVCEVAADEGPPRAAPLHTPHAHHVHHGPHLAHRHRHHRRTLSLDNRDSVDLAELVPAVGAHPRAAWRAAGPAPGHEDCNGRMPSIAKDVFTKMGDRGDRGEDEEEIDYTLCFRVRKMIDVYKPDWCEVREDWSVYLFSPENRFRVLCQTIIAHKLFDYVVLAFIFLNCITIALERPQIEAGSTERIFLTVSNYIFTAIFVGEMTLKVVSLGLYFGEQAYLRSSWNVLDGFLVFVSIIDIVVSLASAGGAKILGVLRVLRLLRTLRPLRVISRAPGLKLVVETLISSLKPIGNIVLICCAFFIIFGILGVQLFKGKFYHCLGVDTRNITNRSDCMAANYRWVHHKYNFDNLGQALMSLFVLASKDGWVNIMYNGLDAVAVDQQPVTNHNPWMLLYFISFLLIVSFFVLNMFVGVVVENFHKCRQHQEAEEARRREEKRLRRLEKKRRKAQRLPYYATYCHTRLLIHSMCTSHYLDIFITFIICLNVVTMSLEHYNQPTSLETALKYCNYMFTTVFVLEAVLKLVAFGLRRFFKDRWNQLDLAIVLLSVMGITLEEIEINAALPINPTIIRIMRVLRIARVLKLLKMATGMRALLDTVVQALPQVGNLGLLFMLLFFIYAALGVELFGKLVCNDENPCEGMSRHATFENFGMAFLTLFQVSTGDNWNGIMKDTLRDCTHDERSCLSSLQFVSPLYFVSFVLTAQFVLINVVVAVLMKHLDDSNKEAQEDAEMDAELELEMAHGLGPGPRLPTGSPGAPGRGPGGAGGGGDTEGGLCRRCYSPAQVGRGWRGARVARGGAPQGQMGSRAREALHRDLCLSGPHPAQLHRRGDPVMRGSVPGHGRREPTGRAAVSCPPHSPGTRLIIPPVPTPSPSA

SEQ ID NO: 5 - DNA, 20 bp, Artificial Sequence
  Oligonucleotide probe
gcctgcccaggtgggcaggg

SEQ ID NO: 6 - DNA, 20 bp, Artificial Sequence
  Oligonucleotide probe
gtccgcctaggagaacctgt

SEQ ID NO: 7 - protein, 10 aa, Homo sapiens
YSPAQVGRGW

SEQ ID NO: 8 - DNA, 26 bp, Artificial Sequence
  Oligonucleotides
cttgtgccggcgctgctactcgcctg

SEQ ID NO: 9 - DNA, 27 bp, Artificial Sequence
  Oligonucleotides
aggatggacgaggacctgtctgagttc

SEQ ID NO: 10 - DNA, 65 bp, Artificial Sequence
  Oligonucleotides
atcgaaattaatacgactcactatagggagacccaagctgatggctgagagcgcctccccgccct

SEQ ID NO: 11 - DNA, 40 bp, Artificial Sequence
  Oligonucleotides
cttggccttgcgcaggatgtggcagacatactggaagatc

SEQ ID NO: 12 - DNA, 40 bp, Artificial Sequence
  Oligonucleotides
gatcttccagtatgtctgccacatcctgcgcaaggccaag

SEQ ID NO: 13 - DNA, 40 bp, Artificial Sequence
  Oligonucleotides
agcccagggccaccagcatggggtccggctgcagtgagag

SEQ ID NO: 14 - DNA, 40 bp, Artificial Sequence
  Oligonucleotides
ctctcactgcagccggaccccatgctggtggccctgggct

SEQ ID NO: 15 - DNA, 40 bp, Artificial Sequence
  Oligonucleotides
ttggtcacaggctgctggtccacagcaacagcatccagtc

SEQ ID NO: 16 - DNA, 40 bp, Artificial Sequence
  Oligonucleotides
gactggatgctgttgctgtggaccagcagcctgtgaccaa

SEQ ID NO: 17 - DNA, 69 bp, Artificial Sequence
  Oligonucleotides
ctagaaggcacagtcgaggctgatcagcgggtttaaactcactactaggcggacggggacggggtgggg

SEQ ID NO: 18 - DNA, 64 bp, Artificial Sequence
  Oligonucleotides
atacgactcactatagggagacccaagctggctagttaagtatgtttatatatggattttgaaa

SEQ ID NO: 19 - DNA, 41 bp, Artificial Sequence
  Oligonucleotides
gtagaggtatggccggtggtgaacatcaccacagaattaac

SEQ ID NO: 20 - DNA, 73 bp, Artificial Sequence
  Oligonucleotides
tccaccacactggactagtggatccgagctcggtaccaaggcccgggcatgctacgtacctgtttaactcttc

SEQ ID NO: 21 - DNA, 41 bp, Artificial Sequence
  Oligonucleotides
gttaattctgtggtgatgttcaccaccggccatacctctac

SEQ ID NO: 22 - DNA, 80 bp, Artificial Sequence
  Oligonucleotide linker
tgcgttatcccctgattctgtggataaccgtattaccgctgggtaataactgatataattaaattgaagctctaatttgt

SEQ ID NO: 23 - DNA, 80 bp, Artificial Sequence
  Oligonucleotide linker
acaaattagagcttcaatttaattatatcagttattacccagcggtaatacggttatccacagaatcaggggataacgca

SEQ ID NO: 24 - DNA, 80 bp, Artificial Sequence
  Oligonucleotide linker
aatttaaattataattatttttatagcacgtgatgaaaagagcatggatctcggggacgtctaactactaagcgagagta

SEQ ID NO: 25 - DNA, 80 bp, Artificial Sequence
  Oligonucleotide linker
tactctcgcttagtagttagacgtccccgagatccatgctcttttcatcacgtgctataaaaataattataatttaaatt

SEQ ID NO: 26 - DNA, 4615 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccaattctctaaggaaatacttaaccatggtcgactggatccggtaccgaattcgcttactaaaagccagataacagtatgcgtatttgcgcgctgatttttgcggtataagaatatatactgatatgtatacccgaagtatgtcaaaaagaggtgtgcttctagaatgcagtttaaggtttacacctataaaagagagagccgttatcgtctgtttgtggatgtacagagtgatattattgacacgcccgggcgacggatagtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatatagaattcgcggccgcactcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgct

SEQ ID NO: 27 - DNA, 88 bp, Artificial Sequence
  oligonucleotide linker
actttgtacaaaaaagcaggcttcgaaggagatagaaccagcccgggcgccgcactcgagatatctagacccagctttcttgtacaaa

SEQ ID NO: 28 - DNA, 88 bp, Artificial Sequence
  oligonucleotide linker
tttgtacaagaaagctgggtctagatatctcgagtgcggcgcccgggctggttctatctccttcgaagcctgcttttttgtacaaagt

SEQ ID NO: 29 - DNA, 4146 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagcccgggcgccgcactcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgct

SEQ ID NO: 30 - DNA, 80 bp, Artificial Sequence
  oligonucleotide linker
gttaaccgggctgcatccgatgcaagtgtgtcgctgtcgagggtaataactgatataattaaattgaagctctaatttgt

SEQ ID NO: 31 - DNA, 80 bp, Artificial Sequence
  oligonucleotide linker
acaaattagagcttcaatttaattatatcagttattaccctcgacagcgacacacttgcatcggatgcagcccggttaac

SEQ ID NO: 32 - DNA, 80 bp, Artificial Sequence
  oligonucleotide linker
ataaaatcattatttgccatccagctgcagctctggcccgtcgaatttctgccattcatccgcttattatcacttattca

SEQ ID NO: 33 - DNA, 80 bp, Artificial Sequence
  oligonucleotide linker
tgaataagtgataataagcggatgaatggcagaaattcgacgggccagagctgcagctggatggcaaataatgattttat

SEQ ID NO: 34 - DNA, 9430 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagcccgggcgccgcactcgagatatctagacccagctttcttgtacaaagttggcattataagaaagcattgcttatcaatttgttgcaacgaacaggtcactatcagtcaaaataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 35 - DNA, 80 bp, Artificial Sequence
  oligonucleotide linker
acggttatccacagaatcaggggataacgcaggaaagaacataaaatcattatttgccatccagctgcagctctggcccg

SEQ ID NO: 36 - DNA, 80 bp, Artificial Sequence
  oligonucleotide linker
cgggccagagctgcagctggatggcaaataatgattttatgttctttcctgcgttatcccctgattctgtggataaccgt

SEQ ID NO: 37 - DNA, 80 bp, Artificial Sequence
  oligonucleotide linker
aatttaaattataattatttttatagcacgtgatgaaaagtccgcgcacatttccccgaaaagtgccacctgacgtcgac

SEQ ID NO: 38 - DNA, 80 bp, Artificial Sequence
  oligonucleotide linker
gtcgacgtcaggtggcacttttcggggaaatgtgcgcggacttttcatcacgtgctataaaaataattataatttaaatt

SEQ ID NO: 39 - DNA, 12604 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 40 - DNA, 41 bp, Artificial Sequence
  oligonucleotides
gtagaggtatggccggtggtcaacatcaccacagaattaac

SEQ ID NO: 41 - DNA, 41 bp, Artificial Sequence
  oligonucleotides
gttaattctgtggtgatgttgaccaccggccatacctctac

SEQ ID NO: 42 - DNA, 14561 bp, Artificial Sequence
  vector
catggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagtatgtttatatatggattttgaaaatcttttaaaaaagcgataaaagcgttgggatctgccactggtaacttcaaaatagacaagacgaaaaaacggtgaaaatgggtgatagaaataatacagaagtagatgttgaattagattaaactgaagatatataatttattggaaaatacatagagtttttgttgatgcgcttaagcgatcaattcaacaacaccaccagcagctctgattttttcttcagccaacttggagacgaatctagctttgacgataactggaacatttggaattctacccttacccaagatcttaccgtaaccggctgccaaagtgtcaataactggagcagtttccttagaagcagatttcaagtattggtctctcttgtcttctgggatcaatgtccacaatttgtccaagttcaagactggcttccagaaatgagcttgttgcttgtggaagtatctcataccaaccttaccgaaataacctggatggtatttatccatgttaattctgtggtgatgttcaccaccggccatacctctaccaccggggtgctttctgtgcttaccgatacgacctttaccggctgtacaaaaaaaatattgtaatgaaatacaatttttaaatcgttagtaaaacaggttttcaactggacattttatcgaaacaaaagaaacgtggtaactctttttcataattgcgctctctacaaccatttgatacagcgataattagtgctttccataatctaaatgaaagttaccttttatagccgtaaaatgagtagacagtatgactgttctgaatatcatggtataataaaggagaagtatgacaaaggctatgcacactttctttttgctcagtttgcgatggaagagtacatttactcgagctattacctcaaataagccgtctcaacagtgagatggtaatacaaagttcgcaatcaaaacacaaagaaatatgttgagtacacttagctgattgacattctcctgtaccatgatatacacacgacatattggttgcacaacactaaaaaaaaagtcaccgtcttggttctttcattccctcttccaaatggaactacatacctgagacgtgacctctgtgctttctagtcttagtgaatctggaaggcattcttgattagttggatgattgttctgggatttaatgcaaaaaaatcactaagaaggaaaaaaatcaacggagaaagcaaacgccatcttaaatatacgggatacagatgaaaggtttgaacctatctgggaaaatacgcattaaacaagcgaaaaactgcgaggaaaattgtttgcgtctctgcgggctattcacgcgccagaggaaaaataggaaaaataacaggcattagaaaaataattttgattttggtaatgtgtgggtccctggtgtacagatgttacattggttacagtactcttgtttttgctgtgtttttcgaaagggtttcctgggaactagtgctataatgtcaacatcaagtatcgtagatttcaattcaacttagctcttattacagtttatatataatacgcatttcataaggttcattccacctcgtctcttgtaatcattacaatgctgttgatcaactatatttaacaatattatagcttgtttctttataacattagcttacgctcataggcggcagtaaacgatgattaaccatgaaacgtaaggtaacggtggcataatttggtatttgttttctttttttcattttttgaactattacttggttttttcgagagcaaactacctcatacttatttcttgtgtgttaatattatattggcggcaaagtgaaaggggtgatcattctgttatgaaaaaaataaagaacttaagaggaacggtgaagaaagaaaacggataaaatgagaagagttaaacaggtacgtagcatgcccgggccttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagct

SEQ ID NO: 43 - DNA, 65 bp, Artificial Sequence
  oligonucleotides
ataaaatcattatttgccatccagctgcagctctggcccgtgtctcaaaatctctgatgttacat

SEQ ID NO: 44 - DNA, 65 bp, Artificial Sequence
  oligonucleotides
tttctctgtccttcctgtgcgacggttacgccgctccatggtctgacgctcagtggaacggggcc

SEQ ID NO: 45 - DNA, 14567 bp, Artificial Sequence
  vector
catggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagtatgtttatatatggattttgaaaatcttttaaaaaagcgataaaagcgttgggatctgccactggtaacttcaaaatagacaagacgaaaaaacggtgaaaatgggtgatagaaataatacagaagtagatgttgaattagattaaactgaagatatataatttattggaaaatacatagagtttttgttgatgcgcttaagcgatcaattcaacaacaccaccagcagctctgattttttcttcagccaacttggagacgaatctagctttgacgataactggaacatttggaattctacccttacccaagatcttaccgtaaccggctgccaaagtgtcaataactggagcagtttccttagaagcagatttcaagtattggtctctcttgtcttctgggatcaatgtccacaatttgtccaagttcaagactggcttccagaaatgagcttgttgcttgtggaagtatctcataccaaccttaccgaaataacctggatggtatttatccatgttaattctgtggtgatgttcaccaccggccatacctctaccaccggggtgctttctgtgcttaccgatacgacctttaccggctgtacaaaaaaaatattgtaatgaaatacaatttttaaatcgttagtaaaacaggttttcaactggacattttatcgaaacaaaagaaacgtggtaactctttttcataattgcgctctctacaaccatttgatacagcgataattagtgctttccataatctaaatgaaagttaccttttatagccgtaaaatgagtagacagtatgactgttctgaatatcatggtataataaaggagaagtatgacaaaggctatgcacactttctttttgctcagtttgcgatggaagagtacatttactcgagctattacctcaaataagccgtctcaacagtgagatggtaatacaaagttcgcaatcaaaacacaaagaaatatgttgagtacacttagctgattgacattctcctgtaccatgatatacacacgacatattggttgcacaacactaaaaaaaaagtcaccgtcttggttctttcattccctcttccaaatggaactacatacctgagacgtgacctctgtgctttctagtcttagtgaatctggaaggcattcttgattagttggatgattgttctgggatttaatgcaaaaaaatcactaagaaggaaaaaaatcaacggagaaagcaaacgccatcttaaatatacgggatacagatgaaaggtttgaacctatctgggaaaatacgcattaaacaagcgaaaaactgcgaggaaaattgtttgcgtctctgcgggctattcacgcgccagaggaaaaataggaaaaataacaggcattagaaaaataattttgattttggtaatgtgtgggtccctggtgtacagatgttacattggttacagtactcttgtttttgctgtgtttttcgaaagggtttcctgggaactagtgctataatgtcaacatcaagtatcgtagatttcaattcaacttagctcttattacagtttatatataatacgcatttcataaggttcattccacctcgtctcttgtaatcattacaatgctgttgatcaactatatttaacaatattatagcttgtttctttataacattagcttacgctcataggcggcagtaaacgatgattaaccatgaaacgtaaggtaacggtggcataatttggtatttgttttctttttttcattttttgaactattacttggttttttcgagagcaaactacctcatacttatttcttgtgtgttaatattatattggcggcaaagtgaaaggggtgatcattctgttatgaaaaaaataaagaacttaagaggaacggtgaagaaagaaaacggataaaatgagaagagttaaacaggtacgtagcatgcccgggccttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagac

SEQ ID NO: 46 - DNA, 15 bp, Artificial Sequence
  oligonucleotides
gccggcgctgctact

SEQ ID NO: 47 - DNA, 17 bp, Artificial Sequence
  oligonucleotides
gacgctgtccagccaca

SEQ ID NO: 48 - DNA, 15 bp, Artificial Sequence
  oligonucleotide probe
ctgcccaggagaacc

SEQ ID NO: 49 - DNA, 20 bp, Artificial Sequence
  oligonucleotides
gctgataatcccgcctgtcc

SEQ ID NO: 50 - DNA, 25 bp, Artificial Sequence
  oligonucleotides
tgatgattaaagagacgctgtccag

SEQ ID NO: 51 - DNA, 19 bp, Artificial Sequence
  oligonucleotide probe
cgcctaggagaacctgtgg

SEQ ID NO: 52 - DNA, 15 bp, Artificial Sequence
  oligonucleotides
gccggcgctgctact

SEQ ID NO: 53 - DNA, 18 bp, Artificial Sequence
  oligonucleotides
ggtcagctccccctccaa

SEQ ID NO: 54 - DNA, 14 bp, Artificial Sequence
  oligonucleotide probe
ctgcccaggactcc

(19) United States
(12) Patent Application Publication
(10) Pub. No.: US 2006/0014251 A1
(43) Pub. Date: Jan. 19, 2006

(54) Alternatively spliced isoform of calcium channel, voltage dependent, alpha-II subunit (CACNA1I)

(76) Inventor: Christopher D. Armour, Kirkland, WA (US)

Correspondence Address: R. Douglas Bradley;Merck & Co., Inc., Patent Department RY60-30, P.O. Box 2000, Rahway, NJ, 07065-0907, US

(21) Appl. No.: 11/180,074

(22) Filed: Jul. 12, 2005

Related U.S. Application Data
(60) Provisional application No. 60/588,131, filed on Jul. 15, 2004.

(51) Int. Cl. G01N 33/53; C07H 21/04; C12P 21/06; C12N 15/09; C07K 14/705

(52) U.S. Cl. 435/69.1; 435/320.1; 435/325; 530/350; 536/23.5; 435/7.1

(57) ABSTRACT

The present invention features nucleic acids and polypeptides encoding a novel splice variant isoform of calcium channel, voltage dependent, alpha-1I subunit (CACNA1I). The polynucleotide sequence of CACNA1Isv1 is provided by SEQ ID NO 3. The amino acid sequence of CACNA1Isv1 is provided by SEQ ID NO 4. The present invention also provides methods for using CACNA1Isv1 polynucleotides and proteins to screen for compounds that bind to CACNA1Isv1.

This application claims priority to U.S. Provisional Patent Application Ser. No. 60/588,131 filed on Jul. 15, 2004, which is incorporated by reference herein in its entirety.

### BACKGROUND OF THE INVENTION

Voltage-gated calcium channels mediate the influx of calcium ions in response to changes in membrane potential in electrically excitable cells such as neurons and myocytes. Calcium is an important second messenger in muscle contraction, chemotaxis, gene expression, synaptic transmission, and secretion of hormones and neurotransmitters. Its entry into the cell can also depolarize the cell membrane, activating other voltage-gated ion channels (reviewed in Perez-Reyes, 2003, Physiol. Rev. 83:117-161; Catterall, 2000, Annu. Rev. Cell Dev. Biol. 16:521-555).

To date ten genes encoding pore-forming α₁ subunits of voltage-gated calcium channels have been identified. These ten genes are grouped into three subfamilies according to their predicted amino acid sequences; these subfamily divisions also coincide with their pharmacological and biophysical properties. The Caᵥ1 subfamily consists of Caᵥ1.1-1.4 (also known as α_{1S, C, D, F}), the Caᵥ2 subfamily: Caᵥ2.1-2.3 (also known as α_{1A, B, E}), the Caᵥ3 subfamily: Caᵥ3.1-3.3 (α_{1G, H, I}). The predicted amino acid sequences of the α₁ subunits have 70% identity within a subfamily but less than 40% identity among subfamilies (Ertel et al., 2000, Neuron 25:533-535).

Caᵥ1 subfamily channels mediate L-type Ca²⁺ currents. Caᵥ2 subfamily mediates P/Q-type, N-type, and R-type currents, and the Caᵥ3 subfamily mediates T-type currents. L-type currents are long-lasting and require a strong depolarization for activation. L-type channels are blocked by organic antagonists such as dihydropyridines, phenyl alkylamines, and benzothiazepines. They are expressed in muscle and endocrine cells, where they mediate contraction and secretion. N-type, P/Q-type, and R-type calcium channels also require strong depolarization. However, they are resistant to L-type channel inhibitors but sensitive to toxins from snails and spiders. These channels are expressed in neurons, initiating neurotransmission at fast synapses. T-type channels have a low voltage threshold and have a fast time course. T-type channels are also resistant to the L-type organic antagonists and snake and spider toxins which discriminate N-, P/Q-, and R-type channels. T-type channels have been found in a wide variety of cell types, including nervous tissue, kidney, heart, smooth muscle, sperm, and endocrine organs and are implicated in neuronal firing, hormone secretion, smooth muscle contraction, myoblast fusion, and fertilization (reviewed in Catterall, 2000, Annu. Rev. Cell Dev. Biol. 16:521-555; Perez-Reyes, 2003, Physiol. Rev. 83:117-161; Ertel et al., 2000, Neuron 25:533-535).

Properties of T-type channels include: opening after small depolarizations of the plasma membrane (low-voltage activated (LVA)); transient currents during a sustained pulse; slow closing upon membrane repolarization, producing slow tail currents; tiny single channel conductance of Ba²⁺ and Ca²⁺; insensitivity to dihydropyridines; and similar voltage range for both activation and steady-state inactivation (reviewed in Perez-Reyes, 2003, Physiol. Rev. 83:117-161). T-currents generated by CACNA1I subunits have slow activation and inactivation kinetics distinct from CACNA1G and CACNA1H (Lee et al., 1999, J. Neurosci. 19:1912-1921; Monteil et al., 2000, J. Biol. Chem. 275:16530-16535).

Calcium channels are complex proteins consisting of multiple subunits. The largest subunit, α₁, contains the conduction pore, voltage sensor, gating apparatus, and sites of channel regulation by second messengers, drugs, and toxins. The α₁ subunit has four homologous domains (Domains I to IV), each containing six α-helical transmembrane segments (S1 to S6), and a membrane-associated loop between S5 and S6 which forms the pore lining of the channel. The S4 segment serves as the voltage sensor, initiating conformational change and opening the pore upon depolarization. β, γ, and α₂δ subunits are auxiliary subunits that modulate the channel's properties (reviewed in Catterall, 2000, Annu. Rev. Cell Dev. Biol. 16:521-555). The specific subunit structures of T-type channels are unknown, as native channels have not yet been purified (reviewed in Perez-Reyes, 2003, Physiol. Rev. 83:117-161).

The human Caᵥ3.3 gene, also known as CACNA1I, has been identified. PCR screening of a brain cDNA library and comparison with genomic sequences revealed a 36 exon gene encoding 2,016 amino acids, located on chromosome 22. The putative extracellular loops contain 5 predicted N-glycosylation sites, and the cytoplasmic portions contain 28 potential phosphorylation sites. Seventeen extracellular cysteines may contribute to proper protein conformation. The transmembrane segments of CACNA1I have 84% identity to the transmembrane segments of the two other T-type channels, CACNA1G and CACNA1H (Mittman et al., 1999, Neurosci. Lett. 269:121-124). Monteil et al. (2000, J. Biol. Chem. 275:16530-16535) also cloned the CACNA1I gene from a brain cDNA library. The cloned gene encoded a 1,981 amino acid protein, and mRNA transcripts were detected in the brain, adrenal and thyroid glands. The CACNA1I gene cloned by Gomora et al. (Biophys. J., 2002, 83:229-241) differs from those reported by Mittman et al. (1999, Neurosci. Lett. 269:121-124) and Monteil et al. (2000, J. Biol. Chem. 275:16530-16535). The predicted 2,188 amino acid protein has an additional 4 kb exon (exon 37) that adds 214 amino acids to the carboxyl terminus compared to the previously reported sequences.

The CACNA1I gene has also been identified in rat. The rat gene encodes a 1,835 amino acid protein that has 93% identity to the human CACNA1I amino acid sequence (Lee et al., 1999, J. Neurosci. 19:1912-1921; Mittman et al., 1999, Neurosci. Lett. 269:121-124).

Splice variants of human CACNA1I have been identified (see U.S. Pat. No. 6,309,858; U.S. Pat. No. 6,589,787; and PCT Publication Number WO 00/70044). Mittman et al. (Neurosci. Lett., 1999, 269:121-124) noted alternative splicing occurring at cassette exon 9 (encoding 35 amino acids) and at an alternative acceptor in exon 33, eliminating 13 amino acids. Chemin et al. (2001, Eur. J. Neurosci. 14:1678-1686) discovered that the use of the internal acceptor site in exon 33 was also associated with the presence of an additional alanine residue in exon 36, due to the use of an alternative 5′ splice acceptor site. Alternative splicing of exon 36 has also been described (PCT Publication Number WO00/70044). Some CACNA1I isoforms are cleaved and polyadenylated just 3′ of exon 36, while others use a donor splice site internal to exon 36 and an acceptor site 5′ to exon 37, adding an additional 214 amino acids. Variants with alternative splicing in exons 33 and 34, encoding distinct carboxy tennini, have also been identified in rat CACNA1I (Murbartian et al., 2002, FEBS Lett. 528:272-278). CACNA1I isoforms with distinct C-terminal regions have demonstrated different current kinetics (Chemin et al., 2001, Eur. J. Neurosci. 14:1678-1686; Murbartian et al., 2002, FEBS Lett. 528:272-278; Gomora et al., 2002, Biophys. J. 83:229-241).

While few mutations in human Caᵥ3 subfamily genes have been described, mutations in other calcium channel genes have been associated with ataxic and epileptic disorders (Jen, 1999, Curr. Opin. Neurobiol. 9:274-280; Kullmann et al., 2002, Brain 125:1177-1195). CACNA1I gene knockout studies have not yet been reported. However, study of knockout mice for another T-type channel, CACNA1G, has provided evidence for the role of T-type channels in the generation of absence seizures (Kim et al., 2001, Neuron 31:35-45). Khosravani et al. (2004, J. Biol. Chem. 279:9681-9684) demonstrated that several mutations in CACNA1H associated with childhood absence epilepsy show greater calcium influx, which may increase propensity for seizures.

In contrast to high voltage-activated calcium channels, T-type channels are relatively resistant to organic calcium channel blockers and peptide toxins. While compounds that inhibit T-type channels have been identified, none of these compounds are highly selective for T-type channels (reviewed in Heady et al., 2001, Jpn. J. Pharmacol. 85: 339-350). CACNA1H channels are sensitive to low concentrations of nickel, but much higher concentrations are required to half-block CACNA1G and CACNA1I (Lee et al., 1999, Biophys. J. 77:3034-3042). Mibefradil, an antihypertensive agent, has been shown to block T-type channels with a 13-fold greater affinity than the high voltage-activated L-type channel (Monteil et al., 2000, J. Biol. Chem. 275:16530-16535; Martin et al., 2000, J. Pharmacol. Exp. Ther. 295:302-308; reviewed in Heady et al., 2001, Jpn. J. Pharmacol. 85:339-350). The endogenous cannabinoid, anandamide, directly inhibits T-type channels, with CACNA1I displaying the most marked modulation compared to CACNA1G and CACNA1H (Chemin et al., 2001, EMBO J. 20:7033-7040). While kurotoxin and kurotoxin-like peptides from scorpion species inhibit T-type calcium channels, they also display cross-reactivity with voltage-gated calcium channels (Chuang et al., 1998, Nat. Neurosci. 1:668-674; Olamendi-Portugal et al., 2002, Biochem. Biophys. Res. Commun. 299:562-568). Succinimide anti-epileptic drugs are also capable of inhibiting calcium T-type channels (Gomora et al., 2001, Mol. Pharmacol. 60:1121-1132).

Calcium T-type channel function is implicated in slow wave sleep and absence epilepsy. Within thalamic neurons, T-type channels are activated by depolarization from hyperpolarized membrane potentials and generate a low threshold calcium spike, which triggers an oscillatory fire mode called burst firing. Low threshold burst firing is thought to underlie the thalamocortical rhythmic oscillations during deep sleep and absence epilepsy (reviewed in Pape et al., 2004, Pflugers Arch. 448:131-138; Perez-Reyes, 2003, Physiol. Rev. 83:117-161). In a study of the rat model of absence epilepsy, a selective increase in T-type calcium conductance of reticular thalamic neurons was observed in affected rats compared to seizure-free rats (Tsakiridou et al., 1995, J. Neurosci. 15:3110-3117). Supporting the hypothesis that T-type channels are involved in thalamocortical dysrhythmias disorders, drugs used as antiepileptics and anesthesics demonstrate T-type channel block (reviewed in Perez-Reyes, 2003, Physiol. Rev. 83:117-161). Ethanol, which is known to disrupt normal sleep rhythms, has been found to affect calcium currents in thalamic relay cells (Mu et al., 2003, J. Pharmacol. Exp. Ther. 307:197-204).

Because of the multiple therapeutic values of drugs targeting calcium channels, including CACNA1I there is a need in the art for compounds that selectively bind to isoforms of CACNA1I. The present invention is directed toward a novel CACNA1I isoform (CACNA1Isv1) and uses thereof.

### SUMMARY OF THE INVENTION

RT-PCR, DNA sequence analysis, and real-time quantitative PCR have been used to identify and confirm the presence of novel splice variants of human CACNA1I mRNA. More specifically, the present invention features polynucleotides encoding different protein isoforms of CACNA1I. A polynucleotide sequence encoding CACNA1Isv1 is provided by SEQ ID NO 3. An amino acid sequence for CACNA1Isv1 is provided by SEQ ID NO 4.

Thus, a first aspect of the present invention describes a purified CACNA1Isv1 encoding nucleic acid. The CACNA1Isv1 encoding nucleic acid comprises SEQ ID NO 3 or the complement thereof. Reference to the presence of one region does not indicate that another region is not present. For example, in different embodiments the inventive nucleic acid can comprise, consist, or consist essentially of an encoding nucleic acid sequence of SEQ ID NO 3.

Another aspect of the present invention describes a purified CACNA1Isv1 polypeptide that can comprise, consist or consist essentially of the amino acid sequence of SEQ ID NO 4.

Another aspect of the present invention describes expression vectors. In one embodiment of the invention, the inventive expression vector comprises a nucleotide sequence encoding a polypeptide comprising, consisting, or consisting essentially of SEQ ID NO 4, wherein the nucleotide sequence is transcriptionally coupled to an exogenous promoter.

Alternatively, the nucleotide sequence comprises, consists, or consists essentially of SEQ ID NO 3, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 3, or a nucleotide sequence encoding a polypeptide comprising, consisting, or consisting essentially of an amino acid sequence of SEQ ID NO 4, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing CACNA1Isv1 polypeptide comprising SEQ ID NO 4. The method involves the step of growing a recombinant cell containing an inventive expression vector under conditions wherein the polypeptide is expressed from the expression vector.

Another aspect of the present invention features a purified antibody preparation comprising an antibody that binds selectively to CACNA1Isv1 as compared to one or more calcium channel isoform polypeptides that are not CACNA1Isv1.

Another aspect of the present invention provides a method of screening for a compound that binds to CACNA1Isv1, or fragments thereof. In one embodiment, the method comprises the steps of: (a) expressing a polypeptide comprising the amino acid sequence of SEQ ID NO 4 or a fragment thereof from recombinant nucleic acid; (b) providing to said polypeptide a labeled CACNA1I ligand that binds to said polypeptide and a test preparation comprising one or more test compounds; (c) and measuring the effect of said test preparation on binding of said test preparation to said polypeptide comprising SEQ ID NO 4.

In another embodiment of the method, a compound is identified that binds selectively to CACNA1Isv1 polypeptide as compared to one or more calcium channel isoform polypeptides that are not CACNA1Isv1. This method comprises the steps of: providing a CACNA1Isv1 polypeptide comprising SEQ ID NO 4; providing a calcium channel isoform polypeptide that is not CACNA1Isv1; contacting said CACNA1Isv1 polypeptide and said calcium channel isoform polypeptide that is not CACNA1Isv1 with a test preparation comprising one or more test compounds; and determining the binding of said test preparation to said CACNA1Isv1 polypeptide and to said calcium channel isoform polypeptide that is not CACNA1Isv1, wherein a test preparation that binds to said CACNA1Isv1 polypeptide but does not bind to said calcium channel isoform polypeptide that is not CACNA1Isv1 contains a compound that selectively binds said CACNA1Isv1 polypeptide.

In another embodiment of the invention, a method is provided for screening for a compound able to bind to or interact with a CACNA1Isv1 protein or a fragment thereof comprising the steps of: expressing a CACNA1Isv1 polypeptide comprising SEQ ID NO 4 or a fragment thereof from a recombinant nucleic acid; providing to said polypeptide a labeled CACNA1I ligand that binds to said polypeptide and a test preparation comprising one or more compounds; and measuring the effect of said test preparation on binding of said labeled CACNA1I ligand to said polypeptide, wherein a test preparation that alters the binding of said labeled CACNA1I ligand to said polypeptide contains a compound that binds to or interacts with said polypeptide.

Another aspect of the present invention provides a method of screening for a compound that binds to one or more calcium channel isoform polypeptides that are not CACNA1Isv1. This method comprises the steps of: providing a CACNA1Isv1 polypeptide comprising SEQ ID NO 4; providing a calcium channel isoform polypeptide that is not CACNA1Isv1; contacting said CACNA1Isv1 polypeptide and calcium channel isoform polypeptide that is not CACNA1Isv1 with a test preparation comprising one or more test compounds; and determining the binding of said test preparation to said CACNA1Isv1 polypeptide and to said calcium channel isoform polypeptide that is not CACNA1Isv1, wherein a test preparation that binds to said calcium channel isoform polypeptide that is not CACNA1Isv1 but not to said CACNA1Isv1 polypeptide contains a compound that selectively binds said calcium channel isoform polypeptide.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1A illustrates the exon structure of human CACNA1I mRNA corresponding to the known reference form of CACNA1I mRNA (labeled AF393329) and the exon structure corresponding to the inventive splice variant transcript (labeled CACNA1Isv1). FIG. 1B depicts the nucleotide sequences of the exon junctions resulting from the splicing of exon 32 to intron 32 [SEQ ID NO 1] and intron 32 to exon 33 [SEQ ID NO 2], in the case of CACNA1Isv1 mRNA. In FIG. 1B, in the case of the CACNA1Isv1 exon 32-intron 32 splice junction sequence [SEQ ID NO 1], the nucleotides shown in italics represent the 20 nucleotides at the 3′ end of exon 32 and the nucleotides shown in underline represent the 20 nucleotides at the 5′ end of intron 32; and in the case of CACNA1Isv1 intron 32-exon 33 splice junction sequence [SEQ ID NO 2], the nucleotides shown in italics represent the 20 nucleotides at the 3′ end of intron 32 and the nucleotides shown in underline represent the 20 nucleotides at the 5′ end of exon 33.

### DEFINITIONS

Unless defined otherwise, all technical and scientific terms used herein have the meaning commonly understood by one of ordinary skill in the art to which this invention belongs.

As used herein, “CACNA1I” refers to a calcium channel, voltage gated, alpha-1I (AF393329). In contrast, reference to a CACNA1I isoform includes AF393329 and other polypeptide isoform variants of CACNA1I.

As used herein, “CACNA1Isv1” refers to a splice variant isoform of human CACNA1I protein, wherein the splice variant has the amino acid sequence set forth in SEQ ID NO 4 (for CACNA1Isv1).

As used herein, “CACNA1I” refers to polynucleotides encoding CACNA1I.

As used herein, “CACNA1Isv1” refers to polynucleotides that are identical to CACNA1I encoding polynucleotides, except that the sequences represented by intron 32 of the CACNA1I messenger RNA are retained in CACNA1Isv1.

As used herein, “CACNA1Isv1” refers to polynucleotides encoding CACNA1Isv1 having an amino acid sequence set forth in SEQ ID NO 4.

As used herein, a “calcium channel isoform” is any isoform of any calcium channel from any organism, including but not limited to human CACNA1S (Caᵥ1.1), CACNA1C (Caᵥ1.2), CACNA1D (Caᵥ1.3), CACNA1A (Caᵥ2.1), CACNA1B (Caᵥ2.2), CACNA1E (Caᵥ2.3), CACNA1G (Caᵥ3.1), CACNA1H (Caᵥ3.2), and CACNA1I (Caᵥ3.3).

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or intemucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv (scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequence encoding human CACNA1Isv1 that is an alternatively spliced isoform of CACNA1I, and to the amino acid sequence encoding this protein. SEQ ID NO 3 is a polynucleotide sequence representing an exemplary open reading frame that encodes the CACNA1Isv1 protein. SEQ ID NO 4 shows the polypeptide sequence of CACNA1 Isv1.

CACNA1Isv1 polynucleotide sequence encoding a CACNA1Isv1 protein, as exemplified and enabled herein include a number of specific, substantial and credible utilities. For example, CACNA1Isv1 encoding nucleic acids were identified in an mRNA sample obtained from a human source (see Example 1). Such nucleic acids can be used as hybridization probes to distinguish between cells that produce CACNA1Isv1 transcripts from human or non-human cells (including bacteria) that do not produce such transcripts. Similarly, antibodies specific for CACNA1Isv1 can be used to distinguish between cells that express CACNA1Isv1 from human or non-human cells (including bacteria) that do not express CACNA1Isv1.

The importance of CACNA1I as a drug target for disorders featuring thalamocortical dysrhythmias including absence epilepsy and sleep disorders, is evidenced by the selective increase in T-type calcium conductance of reticular thalamic neurons in a genetic absence epilepsy rat model and the channel blocking effects of antiepileptic and anesthetic drugs (Tsakiridou et al., J. Neurosci. 1995, 15:3110-3117; Perez-Reyes, 2003, Physiol. Rev. 83:117-161). Given the potential importance of CACNA1I activity to the therapeutic management of epilepsy or sleep disorders, it is of value to identify CACNA1I isoforms and identify CACNA1I-ligand compounds that are isoform specific, as well as compounds that are effective ligands for two or more different CACNA1I isoforms or calcium channel isoforms. In particular, it may be important to identify compounds that are effective inhibitors of a specific CACNA1I isoform activity, yet do not bind to or interact with a plurality of different CACNA1I isoforms or calcium channel isoforms. Compounds that bind to or interact with multiple CACNA1I isoforms may require higher drug doses to saturate multiple CACNA1I-isoform binding sites and thereby result in a greater likelihood of secondary non-therapeutic side effects. Furthermore, biological effects could also be caused by the interaction of a drug with the CACNA1Isv1 isoform specifically. For the foregoing reasons, CACNA1Isv1 protein represents a useful compound binding target and has utility in the identification of new CACNA1I-ligands and calcium channel isoform-ligands exhibiting a preferred specificity profile and having greater efficacy for their intended use.

In some embodiments, CACNA1Isv1 activity is modulated by a ligand compound to achieve one or more of the following: prevent or reduce the risk of occurrence, or recurrence of thalamocortical dysrhythmia disorders including epilepsy and sleep disorders.

Compounds modulating CACNA1Isv1 include agonists, antagonists, and allosteric modulators. While not wishing to be limited to any particular theory of therapeutic efficacy, generally, but not always, CACNA1Isv1 compounds will be used to modulate the activity of the CACNA1I voltage gated calcium channel. These compounds may act as pore blockers and inhibit the passage of calcium ions across the cellular membrane (Gomora et al., 2001, Mol. Pharmacol. 60:1121-1132). Compounds that bind and stabilize T-channels in the inactivated state may also be utilized (Chemin et al., 2001, EMBO J. 20:7033-7040). Peptides that interact with the voltage-sensing domain and modify channel gating may be used a channel blockers (Chuang et al., 1998, Nature Neurosci. 1:668-674). Calcium channel blocking drugs have been used as anti-arrhythmic drugs and anti-convulsants (reviewed in Heady et al., 2001, Jpn. J. Pharmacol. 85:339-350). Therefore, agents that modulate CACNA1I activity may be used to achieve a therapeutic benefit for any disease or condition due to, or exacerbated by, CACNA1I ion channel activity.

CACNA1Isv1 activity can also be affected by modulating the cellular abundance of transcripts encoding CACNA1Isv1. Compounds modulating the abundance of transcripts encoding CACNA1Isv1 include a cloned polynucleotide encoding CACNA1Isv1 that can express CACNA1Isv1 in vivo, antisense nucleic acids targeted to CACNA1Isv1 transcripts, enzymatic nucleic acids, such as ribozymes, and RNAi nucleic acids, such as shRNAs or siRNAs, targeted to CACNA1Isv1 transcripts.

In some embodiments, CACNA1Isv1 activity is modulated to achieve a therapeutic effect upon diseases in which regulation of CACNA1I is desirable. For example, epilepsy and sleep disorders may be treated by modulating CACNA1Isv1 calcium channel activity.

CACNA1Isv1 Nucleic Acids

CACNA1Isv1 nucleic acids contain regions that encode for polypeptides comprising, consisting, or consisting essentially of SEQ ID NO 4. The CACNA1Isv1 nucleic acids have a variety of uses, such as use as a hybridization probe or PCR primer to identify the presence of CACNA1Isv1 nucleic acids; use as a hybridization probe or PCR primer to identify nucleic acids encoding for proteins related to CACNA1Isv1; and/or use for recombinant expression of CACNA1Isv1 polypeptides. In particular, CACNA1Isv1 polynucleotides have an additional polynucleotide region that comprises intron 32 of the CACNA1I gene.

The invention also encompass splice variants of a nucleotide sequence encoding CACNA1Isv1. A splice variant may have significant identity to a reference molecule, but will generally have greater or lesser number of polynucleotides due to alternate splicing of exons during mRNA processing. The corresponding polypeptide may possess additional functional domains or an absence of domains. For example, a CACNA1I splice variant has been described which possesses additional sequence deriving from exon 9 of the CACNA1I gene (reference transcript NM_{—}021096) (Mittman et al., 1999, Neurosci. Lett. 269:121-124; PCT Publication No. WO 00/70044). In an alternative embodiment, CACNA1Isv1 polynucleotides have additional polynucleotide regions that comprise exon 9 and intron 32 of the CACNA1I gene.

The invention also encompasses a polymorphic variant of a nucleotide sequence encoding CACNA1Isv1. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state. For example, refSNP ID: rs7288746 indicates a SNP located in intron 32 of the CACNA1I gene, consisting of C/T alleles. In another example, refSNP ID: rs136853 identifies an SNP located in exon 17 of the CACNA1I gene, consisting of A/G alleles.

Regions in CACNA1Isv1 nucleic acid that do not encode for CACNA1Isv1, or are not found in SEQ ID NO 3, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding CACNA1Isv1 related proteins from different sources. Obtaining nucleic acids encoding CACNA1Isv1 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

CACNA1Isv1 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=lsoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asn=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

CACNA1Isv1 Probes

Probes for CACNA1Isv1 contain a region that can specifically hybridize to CACNA1Isv1 target nucleic acids, under appropriate hybridization conditions and can distinguish CACNA1Isv1 nucleic acids from each other and from non-target nucleic acids, in particular CACNA1I polynucleotides lacking intron 32. Probes for CACNA1Isv1 can also contain nucleic acid regions that are not complementary to CACNA1Isv1 nucleic acids.

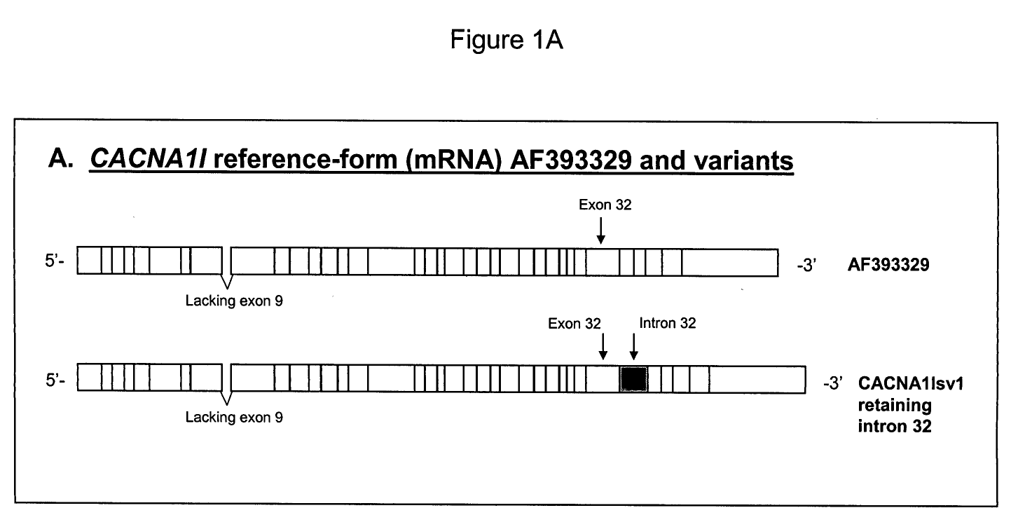

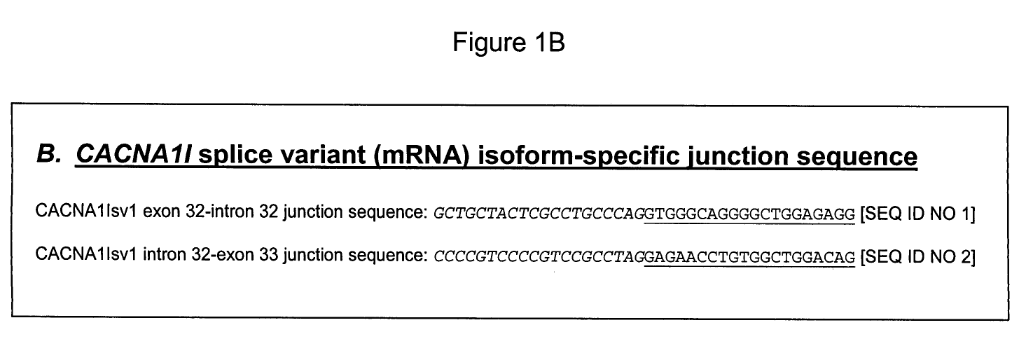

In embodiments where, for example, CACNA1Isv1 polynucleotide probes are used in hybridization assays to specifically detect the presence of CACNA1Isv1 polynucleotides in samples, the CACNA1Isv1 polynucleotides comprise at least 20 nucleotides of the CACNA1Isv1 sequence that correspond to the respective novel exon junction or novel polynucleotide regions. In particular, for detection of CACNA1Isv1, the probe comprises at least 20 nucleotides of the CACNA1Isv1 sequence that corresponds to an exon junction polynucleotide created by the alternative splicing of exon 32 to intron 32 of the primary transcript of the CACNA1I gene (see FIGS. 1A and 1B). For example, the polynucleotide sequence: 5′ GCCTGCCCAGGTGGGCAGGG 3′ [SEQ ID NO 5] represents one embodiment of such an inventive CACNA1Isv1 polynucleotide wherein a first 10 nucleotide region is complementary and hybridizable to the 3′ end of exon 32 of the CACNA1I gene and a second 10 nucleotide region is complementary and hybridizable to the 5′ end of intron 32 of the CACNA1I gene (see FIG. 1B).

In another example, the polynucleotide sequence: 5′ GTCCGCCTAGGAGAACC TGT 3′ [SEQ ID NO 6] represents one embodiment of such an inventive CACNA1Isv1 polynucleotide wherein a first 10 nucleotides region is complementary and hybridizable to the 3′ end of intron 32 of the CACNA1I gene and a second 10 nucleotide region is complementary and hybridizable to the 5′ end of exon 33 of the CACNA1I gene (see FIG. 1B).

In some embodiments, the first 20 nucleotides of a CACNA1Isv1 probe comprise a first continuous region of 5 to 15 nucleotides that is complementary and hybridizable to the 5′ end of exon 32 and a second continuous region of 5 to 15 nucleotides that is complementary and hybridizable to the 5′ end of intron 32 of the CACNA1I gene, or alternatively, the first 20 nucleotides of a CACNA1Isv1 probe comprise a first continuous region of 5 to 15 nucleotides that is complementary and hybridizable to the 3′ end of intron 32 and a second continuous region of 5 to 15 nucleotides that is complementary and hybridizable to the 5′ end of exon 33.

In other embodiments, the CACNA1Isv1 polynucleotide comprises at least 40, 60, 80 or 100 nucleotides of the CACNA1Isv1 sequence, that correspond to a junction polynucleotide region created by the lack of splicing of exon 32 to exon 33 resulting in the retention of intron 32 of the primary transcript of the CACNA1I gene. In embodiments involving CACNA1Isv1, the CACNA1Isv1 polynucleotide is selected to comprise a first continuous region of at least 5 to 15 nucleotides that is complementary and hybridizable to the 3′ end of exon 32 and a second continuous region of at least 5 to 15 nucleotides that is complementary and hybridizable to the 5′ end of intron 32, or the CACNA1Isv1 polynucleotide is selected to comprise a first continuous region of at least 5 to 15 nucleotides that is complementary and hybridizable to the 3′ end of intron 32 and a second continuous region of at least 5 to 15 nucleotides that is complementary and hybridizable to the 5′ end of exon 33 of the CACNA1I gene. As will be apparent to a person of skill in the art, a large number of different polynucleotide sequences from the region of the exon 32 to intron 32 or intron 32 to exon 33 splice junction may be selected which will, under appropriate hybridization conditions, have the capacity to detectably hybridize to CACNA1Isv1 polynucleotides and yet will hybridize to a much less extent or not at all to CACNA1I isoform polynucleotides wherein exon 32 is not spliced to intron 32 or wherein intron 32 is not spliced to exon 33.

Preferably, non-complementary nucleic acid that is present has a particular purpose such as being a reporter sequence or being a capture sequence. However, additional nucleic acid need not have a particular purpose as long as the additional nucleic acid does not prevent the CACNA1Isv1 nucleic acid from distinguishing between target polynucleotides, e.g., CACNA1Isv1 polynucleotides, and non-target polynucleotides, including, but not limited to CACNA1I polynucleotides not comprising exon 32 to intron 32 or intron 32 to exon 33 splice junctions found in CACNA1Isv1.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

CACNA1Isv1 polynucleotides, such as those comprising SEQ ID NO 3, can be used to make CACNA1Isv1 polypeptides. In particular, CACNA1Isv1 polypeptides can be expressed from recombinant nucleic acids in a suitable host or in vitro using a translation system. Recombinantly expressed CACNA1Isv1 polypeptides can be used, for example, in assays to screen for compounds that bind CACNA1Isv1. Alternatively, CACNA1Isv1 polypeptides can also be used to screen for compounds that bind to one or more CACNA1I or calcium channel isoforms, but do not bind to CACNA1Isv1.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pRS416 (ATCC 87521), pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad, Calif.).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

Recombinant DNA molecules that feature precise fusions of polynucleotide sequences can also be assembled using standard recombinational subcloning techniques. Recombination-mediated, PCR-directed, or PCR-independent plasmid construction in yeast is well known in the art (see Hua et al., 1997, Plasmid 38:91-96; Hudson et al., 1997, Genome Res. 7(12):1169-1173; Oldenburg et al., 1997, Nucleic Acids Res. 25(2):451-452; Raymond et al., 1999, BioTechniques 26(1)134-8, 140-1). Overlapping sequences between the donor DNA fragments and the acceptor plasmid permit recombination in yeast. An example of recombination-mediated plasmid construction in Saccharomyces cerevisiae is described in Oldenburg et al., 1997, Nucleic Acids Res. 25(2):451-452: a DNA segment of interest was amplified by PCR so that the PCR product had 20-40 bp of homology at each end to the region of the plasmid at which recombination was to occur. The PCR product and linearized plasmid were co-transformed into yeast, and recombination resulted in replacement of the region between the homologous sequences on the plasmid with the region carried by the PCR fragment. The recombinational method of plasmid construction bypasses the need for extensive modification and ligation steps and does not rely on available restriction sites. These cloning vectors can then be utilized for protein expression in multiple systems.

To enhance expression in a particular host it may be useful to modify the sequence provided in SEQ ID NO 3 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

CACNA1Isv1 Polypeptides

CACNA1Isv1 polypeptides contain an amino acid sequence comprising, consisting or consisting essentially of SEQ ID NO 4. CACNA1Isv1 [SEQ ID NO 4] has 80.6% amino acid sequence identity as compared to the CACNA1I reference protein (AAM67414). CACNA1Isv1 polypeptides have a variety of uses, such as providing a marker for the presence of CACNA1Isv1; use as an immunogen to produce antibodies binding to CACNA1Isv1; use as a target to identify compounds binding selectively to CACNA1Isv1; or use in an assay to identify compounds that bind to one or more CACNA1I or calcium channel isoforms but do not bind to or interact with CACNA1Isv1.

In chimeric polypeptides containing one or more regions from CACNA1Isv1 and one or more regions not from CACNA1Isv1, the region(s) not from CACNA1Isv1 can be used, for example, to achieve a particular purpose or to produce a polypeptide that can substitute for CACNA1Isv1, or fragments thereof. Particular purposes that can be achieved using chimeric CACNA1Isv1 polypeptides include providing a marker for CACNA1Isv1 activity and altering the activity and regulation of the CACNA1I calcium channel.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional CACNA1Isv1

Functional CACNA1Isv1 is a different protein isoform of CACNA1I. The identification of the amino acid and nucleic acid sequences of CACNA1Isv1 provides tools for obtaining functional proteins related to CACNA1Isv1 from other sources, for producing CACNA1Isv1 chimeric proteins, and for producing functional derivatives of SEQ ID NO 4.

CACNA1Isv1 polypeptides can be readily identified and obtained based on their sequence similarity to CACNA1Isv1 [SEQ ID NO 4]. In particular, CACNA1Isv1 polypeptide contains additional amino acids, encoded by nucleotides after the splice junction that results from the retention of intron 32 of the CACNA1I gene. The addition of intron 32 and the splicing of exon 32 to intron 32, and intron 32 to exon 33 of the CACNA1I heteronuclear RNA (hnRNA) transcript does not alter the protein reading frame at the exon 32 to intron 32 and intron 32 to exon 33 splice junctions but introduces a stop codon 3 nucleotides before the intron 32-exon 33 splice junction. Thus, the CACNA1Isv1 polypeptide contains 91 unique amino acids encoded by nucleotides corresponding to intron 32 of the CACNA1I hnRNA and lacks 425 amino acids as compared to the CACNA1I reference protein (AAM67414), which lacks an exon 9 sequence.

Both the amino acid and nucleic acid sequences of CACNA1Isv1 can be used to help identify and obtain CACNA1Isv1 polypeptides. For example, SEQ ID NO 3 can be used to produce degenerative nucleic acid probes or primers for identifying and cloning nucleic acid polynucleotides encoding for a CACNA1Isv1 polypeptide. In addition, polynucleotides comprising, consisting, or consisting essentially of SEQ ID NO 3 or fragments thereof, can be used under conditions of moderate stringency to identify and clone nucleic acids encoding CACNA1Isv1 polypeptides from a variety of different organisms.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with CACNA1Isv1 obtained from a particular source, derivatives can be produced. Such derivatives include polypeptides with amino acid substitutions, additions and deletions. Changes to CACNA1Isv1 to produce a derivative having essentially the same properties should be made in a manner not altering the tertiary structure of CACNA1Isv1.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

CACNA1Isv1 Antibodies

Antibodies recognizing CACNA1Isv1 can be produced using a polypeptide containing SEQ ID NO 4 in the case of CACNA1Isv1, or a fragment thereof as an immunogen. Preferably, a CACNA1Isv1 polypeptide used as an immunogen consists of a polypeptide of SEQ ID NO 4 or a SEQ ID NO 4 fragment having at least 10 contiguous amino acids in length corresponding to the polynucleotide region representing the junction from exon 32 to intron 32 of the CACNA1I gene.

In some embodiments where, for example, CACNA1Isv1 polypeptides are used to develop antibodies that bind specifically to CACNA1Isv1 and not to other isoforms of CACNA1I, the CACNA1Isv1 polypeptides comprise at least 10 amino acids of the CACNA1Isv1 polypeptide sequence corresponding to a junction polynucleotide region created by the alternative splicing of exon 32 to intron 32 of the primary transcript of the CACNA1I gene (see FIG. 1). For example, the amino acid sequence: amino terminus- YSPAQVGRGW -carboxy terminus [SEQ ID NO 7] represents one embodiment of such an inventive CACNA1Isv1 polypeptide wherein a first 5 amino acid region is encoded by a nucleotide sequence at the 3′ end of exon 32 of the CACNA1I gene and a second 5 amino acid region is encoded by the nucleotide sequence at the 5′ end of intron 32. Preferably, at least 10 amino acids of the CACNA1Isv1 polypeptide comprise a first continuous region of 2 to 8 amino acids that is encoded by nucleotides at the 3′ end of exon 32 and a second continuous region of 2 to 8 amino acids that is encoded by nucleotides at the 5′ end of intron 32.

In other embodiments, CACNA1Isv1-specific antibodies are made using a CACNA1Isv1 polypeptide that comprises at least 20, 30, 40 or 50 amino acids of the CACNA1Isv1 sequence that corresponds to a junction polynucleotide region created by the alternative splicing of exon 32 to intron 32 of the primary transcript of the CACNA1I gene. In each case the CACNA1Isv1 polypeptides are selected to comprise a first continuous region of at least 5 to 15 amino acids that is encoded by nucleotides at the 3′ end of exon 32 and a second continuous region of 5 to 15 amino acids that is encoded by nucleotides directly after the novel splice junction created by the splicing of exon 32 to intron 32 of the CACNA1I gene.

Antibodies to CACNA1Isv1 have different uses, such as to identify the presence of CACNA1Isv1 and to isolate CACNA1Isv1 polypeptides. Identifying the presence of CACNA1Isv1 can be used, for example, to identify cells producing CACNA1Isv1. Such identification provides an additional source of CACNA1Isv1 and can be used to distinguish cells known to produce CACNA1Isv1 from cells that do not produce CACNA1Isv1. For example, antibodies to CACNA1Isv1 can distinguish human cells expressing CACNA1Isv1 from human cells not expressing CACNA1Isv1 or non-human cells (including bacteria) that do not express CACNA1Isv1. Such CACNA1Isv1 antibodies can also be used to determine the effectiveness of CACNA1Isv1 ligands, using techniques well known in the art, to detect and quantify changes in the protein levels of CACNA1Isv1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

CACNA1Isv1 Binding Assay

A number of compounds known to modulate calcium channel activity have been disclosed, including mibefradil, kurotoxin, succinimide, and nickel (Martin et al., 2000, J. Pharmacol. Exp. Ther. 295:302-308; Chuang et al., 1998, Nature Neurosci. 1:668-674; Gomora et al., 2001, Mol Pharmacol. 60:1121-1132; Lee et al., 1999, Biophys. J. 77:3034-3042). Methods for expressing calcium channels in Xenopus oocytes and monitoring the activity of these channels, including analyzing the effect of compounds on the activity of calcium channel activity, have been described previously (Lee et al., 1999, Biophys. J. 77:3034-3042; Lee et al., 1999, J. Neurosci. 19:1912-1921; Gomora et al., 2002, Biophys. J. 83: 229-241; Martin et al., 2000, 295:302-308; McRory et al., 276:3999-4011). Methods for screening compounds for their effects on calcium channel activity have also been disclosed (see for example US 2002/0025568, US 2002/0045159, WO 03/006103). A person skilled in the art may use these methods, and others, to screen CACNA1Isv1 polypeptides for compounds that bind to, and in some cases functionally alter, each CACNA1I isoform protein.

CACNA1Isv1, or fragments thereof, can be used in binding studies to identify compounds binding to or interacting with CACNA1Isv1, or fragments thereof. In one embodiment, CACNA1Isv1, or a fragment thereof, can be used in binding studies with a calcium channel isoform protein, or a fragment thereof, to identify compounds that: bind to or interact with CACNA1Isv1 and other calcium channel isoforms; bind to or interact with one or more other calcium channel isoforms and not with CACNA1Isv1; bind to or interact with CACNA1Isv1 and not with one or more other calcium channel isoforms. Such binding studies can be performed using different formats including competitive and non-competitive formats. Further competition studies can be carried out using additional compounds determined to bind to CACNA1Isv1, other CACNA1I isoforms, or other calcium channel isoforms.

The particular CACNA1Isv1 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using CACNA1Isv1 expressed from a recombinant nucleic acid. Alternatively, recombinantly expressed CACNA1Isv1 consists of the SEQ ID NO 4 amino acid sequence.

Binding assays can be performed using individual compounds or preparations containing different numbers of compounds. A preparation containing different numbers of compounds having the ability to bind to CACNA1Isv1 can be divided into smaller groups of compounds that can be tested to identify the compound(s) binding to CACNA1Isv1.

Binding assays can be performed using recombinantly produced CACNA1Isv1 present in different environments. Such environments include, for example, cell extracts and purified cell extracts containing a CACNA1Isv1 recombinant nucleic acid; and also include, for example, the use of a purified CACNA1Isv1 polypeptide produced by recombinant means which is introduced into different environments.

In one embodiment of the invention, a binding method is provided for screening for a compound able to bind selectively to CACNA1Isv1. The method comprises the steps: providing a CACNA1Isv1 polypeptide comprising SEQ ID NO 4; providing a calcium channel isoform polypeptide that is not CACNA1Isv1; contacting the CACNA1Isv1 polypeptide and the calcium channel isoform polypeptide that is not CACNA1Isv1 with a test preparation comprising one or more test compounds; and then determining the binding of the test preparation to the CACNA1Isv1 polypeptide and to the calcium channel isoform polypeptide that is not CACNA1Isv1, wherein a test preparation that binds to the CACNA1Isv1 polypeptide, but does not bind to the calcium channel isoform polypeptide that is not CACNA1Isv1, contains one or more compounds that selectively bind to CACNA1Isv1.

In another embodiment of the invention, a binding method is provided for screening for a compound able to bind selectively to a calcium channel isoform polypeptide that is not CACNA1Isv1. The method comprises the steps: providing a CACNA1Isv1 polypeptide comprising SEQ ID NO 4; providing a calcium channel isoform polypeptide that is not CACNA1Isv1; contacting the CACNA1Isv1 polypeptide and the calcium channel isoform polypeptide that is not CACNA1Isv1 with a test preparation comprising one or more test compounds; and then determining the binding of the test preparation to the CACNA1Isv1 polypeptide and the calcium channel isoform polypeptide that is not CACNA1Isv1, wherein a test preparation that binds the calcium channel isoform polypeptide that is not CACNA1Isv1, but does not bind CACNA1Isv1, contains a compound that selectively binds the calcium channel isoform polypeptide that is not CACNA1Isv1.

The above-described selective binding assays can also be performed with a polypeptide fragment of CACNA1Isv1, wherein the polypeptide fragment comprises at least 10 consecutive amino acids that are coded by a nucleotide sequence that bridges the junction created by the splicing of the 3′ end of exon 32 to the 5′ end of intron 32. Similarly, the selective binding assays may also be performed using a polypeptide fragment of a calcium channel isoform polypeptide that is not CACNA1Isv1, wherein the polypeptide fragment comprises at least 10 consecutive amino acids that are coded by: a) a nucleotide sequence that is contained within intron 32 of the CACNA1I gene; or c) a nucleotide sequence that bridges the junction created by the splicing of the 3′ end of exon 32 to the 5′ end of exon 33 of the CACNA1I gene.

CACNA1I Functional Assays

CACNA1I encodes the alpha subunit of a highly conserved voltage gated calcium channel that is implicated in epilepsy and sleeping disorders. Splice variants of calcium channels may exhibit different current kinetics and different binding affinities for compounds, peptides and other small molecules. The identification of CACNA1Isv1 as a splice variant of CACNA1I provides a means of screening for compounds that bind to CACNA1Isv1 protein thereby altering the activity or regulation of CACNA1Isv1 calcium channels. Assays involving a functional CACNA1sv1 polypeptide can be employed for different purposes, such as selecting for compounds active at CACNA1sv1; evaluating the ability of a compound to affect the ion channel activity of the splice variant; and mapping the activity of different CACNA1Isv1 regions. CACNA1Isv1 activity can be measured using different techniques such as: detecting a change in the intracellular conformation of CACNA1Isv1; detecting a change in the intracellular location of CACNA1Isv1; or measuring the ion channel activity of CACNA1Isv1.

Recombinantly expressed CACNA1Isv1 can be used to facilitate the determination of whether a compound's activity in a cell is dependent upon the presence of CACNA1Isv1. For example, CACNA1Isv1 can be expressed by an expression vector in a cell line and used in a co-culture growth assay, such as described in U.S. Pat. No. 6,518,035, to identify compounds that alter the growth of the cell expressing CACNA1Isv1 from the expression vector as compared to the same cell line but lacking the CACNA1Isv1 expression vector. Alternatively, determination of whether a compound's activity on a cell is dependent upon the presence of CACNA1Isv1 can also be done using gene expression profile analysis methods as described, for example, in U.S. Pat. No. 6,324,479.

Techniques for measuring voltage gated ion channel activity are well known in the art. Methods for expressing calcium channels in Xenopus oocytes and human cells, and monitoring the activity of these channels, including analyzing the effect of compounds on the activity of calcium channel activity, have been described previously (U.S. Pat. No. 6,589,787; Lee et al., 1999, Biophys. J. 77:3034-3042; Lee et al., 1999, J. Neurosci. 19:1912-1921; Gomora et al., 2002, Biophys. J. 83: 229-241; Martin et al., 2000, 295:302-308; McRory et al., 276:3999-4011). The patch clamp technique measures ion current through ion channel proteins and can be used to analyze the effect of drugs on ion channel function. The activity of these channels can be measured electrically (single channel recording) or, alternatively, the patch can be ruptured allowing measurements of the combined channel activity of the entire cell membrane (whole cell recording) (Neher et al., 1978, Pflugers Arch. 375: 219-28; Hamill et al., 1981, Pflugers Arch. 391:85-100; Sakman et al., 1984, Annu Rev Physiol. 46: 455-72; Neher et al., 1992, Sci. Am. 266: 44-51). Other methods for measuring ion channel activity include optical reading of voltage-sensitive dyes (Cohen et al., 1978, Annual Reviews of Neuroscience 1:171-82) and extracellular recording of fast events using metal (Thomas et al., 1972, Exp. Cell Res. 74:61-66) or field effect transistor (Fromherz et al., 1991, Science 252:1290-1293) electrodes. High throughput methods for assaying ion channel activity have also been described (see WO 03/006103 and US 2002/0028480). A variety of other assays has been used to investigate the properties of calcium channels and therefore would also be applicable to the measurement of CACNA1Isv1 function.

CACNA1Isv1 functional assays can be performed using cells expressing CACNA1Isv1 at a high level. These proteins will be contacted with individual compounds or preparations containing different compounds. A preparation containing different compounds where one or more compounds affect CACNA1Isv1 in cells over-producing CACNA1Isv1 as compared to control cells containing an expression vector lacking CACNA1Isv1 coding sequences, can be divided into smaller groups of compounds to identify the compound(s) affecting CACNA1Isv1 activity.

CACNA1Isv1 functional assays can be performed using recombinantly produced CACNA1Isv1 present in different environments. Such environments include, for example, cell extracts and purified cell extracts containing the CACNA1Isv1 expressed from recombinant nucleic acid; and the use of purified CACNA1Isv1 produced by recombinant means that is introduced into a different environment suitable for measuring ion channel activity.

Modulating CACNA1Isv1 Expression

CACNA1Isv1 expression can be modulated as a means for increasing or decreasing CACNA1Isv1 activity. Such modulation includes inhibiting the activity of nucleic acids encoding the CACNA1I isoform target to reduce CACNA1I isoform protein or polypeptide expression, or supplying CACNA1I nucleic acids to increase the level of expression of the CACNA1I target polypeptide thereby increasing CACNA1I activity.

Inhibition of CACNA1Isv1 Activity

CACNA1Isv1 nucleic acid activity can be inhibited using nucleic acids recognizing CACNA1Isv1 nucleic acid and affecting the ability of such nucleic acid to be transcribed or translated. Inhibition of CACNA1Isv1 nucleic acid activity can be used, for example, in target validation studies.

A preferred target for inhibiting CACNA1Isv1 is mRNA stability and translation. The ability of CACNA1Isv1 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding region of a gene that disrupts the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Methods for using RNAi to modify calcium channel activity have been described previously (Keller et al., 2000, J. Pharmacol. Exp. Ther. 295(1):367-72). Modified and unmodified nucleic acids can be used as anti-sense molecules, RNAi and ribozymes. Different types of modifications can affect certain RNA activities such as the ability to be cleaved by RNAse H, and can affect nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing CACNA1Isv1 Expression

Nucleic acids encoding for CACNA1Isv1 can be used, for example, to cause an increase in CACNA1I activity or to create a test system (e.g., a transgenic animal) for screening for compounds affecting CACNA1Isv1 expression. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of CACNA1Isv1 Using RT-PCR

The structure of CACNA1I mRNA in the region corresponding to exons 32 to 34, which corresponds to S6 of Domain IV and a portion of the C-terminus of CACNA1I, was determined for human whole brain using an RT-PCR based assay. Total RNA isolated from human brain was obtained from BD Biosciences Clontech (Palo Alto, Calif.). RT-PCR primers were selected that were complementary to sequences in exon 32 and exon 34 of the reference exon coding sequences in CACNA1I (AF393329). Based upon the nucleotide sequence of CACNA1I mRNA, the CACNA1I exon 32 and exon 34 primer set (hereafter CACNA1I₃₂₋₃₄ primer set) was expected to amplify a 237 base pair amplicon representing the “reference” CACNA1I mRNA region. The CACNA1I exon 32 forward primer has the sequence: 5′ CTTGTGCCGGCGCTGCTACTCGCCTG 3′ [SEQ ID NO 8]; and the CACNA1I exon 34 reverse primer has the sequence: 5′ AGGATGGACGAGGACCTGTCTGAGTTC 3′ [SEQ ID NO 9].

Twenty-five ng of total RNA from whole brain was subjected to a one-step reverse transcription-PCR amplification protocol using the Qiagen, Inc. (Valencia, Calif.), One-Step RT-PCR kit, using the following cycling conditions:

50° C. for 30 minutes;

95° C. for 15 minutes;

35 cycles of:
94° C. for 30 seconds;
63.5° C. for 40 seconds;
72° C. for 50 seconds; then
72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were cloned into an Invitrogen pCR2.1 vector using the reagents and instructions provided with the TOPO TA cloning kit (Invitrogen, Carlsbad, Calif.). Clones were then sequenced from each end (using the same primers used for RT-PCR) by Qiagen Genomics, Inc. (Bothell, Wash.).

At least one different RT-PCR amplicon was obtained from human brain RNA samples using the CACNA1I₃₂₋₃₄ primer set (data not shown). The brain sample assayed exhibited the expected amplicon size of 237 base pairs for normally spliced CACNA1I mRNA. In addition, the brain sample assayed also exhibited an amplicon of about 513 base pairs.

Sequence analysis of the about 513 base pair amplicon revealed that this amplicon form results from the retention of intron 32 of the CACNA1I hnRNA. That is, the longer form CACNA1I amplicon is due to the insertion of an intron 32 polynucleotide sequence. This splice variant form was designated CACNA1Isv1 [SEQ ID NO 3]. Thus, the RT-PCR results suggested that CACNA1I mRNA in brain tissue is composed of a mixed population of molecules wherein at least one of the CACNA1I mRNA splice junctions is altered.

### Example 2

### Cloning of CACNA1Isv1

RT-PCR and sequencing data indicate that in addition to the normal CACNA1I reference mRNA sequence, AF393329, encoding CACNA1I protein, AAM67414, one novel splice variant form of CACNA1I mRNA also exists in brain tissue.

Clones having a nucleotide sequence comprising the splice variant identified in Example 1 (hereafter referred to as CACNA1Isv1) are isolated using recombination-mediated, PCR-directed plasmid construction in yeast. A set of four primer pairs are used to amplify the CACNA1I coding region (AF393329) in four, sequential, overlapping segments (hereafter ORF1, ORF2, ORF3, and ORF4). A 5′ “forward” ORF1 primer and a 3′ “reverse” ORF1 primer, a 5′ “forward” ORF2 primer and a 3′ “reverse” ORF2 primer, a 5′ “forward” ORF3 primer and a 3′ “reverse” ORF3 primer, and a 5′ “forward” ORF4 primer and a 3′ “reverse” ORF4 primer are made and used to amplify and clone the CACNA1I mRNA (AF393329) coding sequences corresponding to ORF1, ORF2, ORF3, and ORF4, respectively.

The 5′ “forward” ORF1 primer is designed to have the nucleotide sequence of 5′ ATCGAAATTAATACGACTCACTATAGGGAGACCCAAGCTGATGGCTGAGAGCGCCTCCCCGCC CT 3′ [SEQ ID NO 10] and to have coordinates 1-25 relative to the CACNA1I mRNA (AF393329). The 3′ “reverse” ORF1 primer is designed to have the nucleotide sequence of 5′ CTTGGCCTTGCGCAGGA TGTGGCAGACATACTGGAAGATC 3′ [SEQ ID NO 11] and to have coordinates 1311-1350 relative to the CACNA1I mRNA (AF393329).

The 5′ “forward” ORF2 primer is designed to have the nucleotide sequence of 5′ GATC TTCCAGTATGTCTGCCACATCCTGCGCAAGGCCAAG 3′ [SEQ ID NO 12] and to have coordinates 1311-1350 relative to the CACNA1I mRNA (AF393329). The 3′ “reverse” ORF2 primer is designed to have the nucleotide sequence of 5′ AGCCCAGGGCCACCAGCATGGGGTCCGGCTGCAGTGAG AG 3′ [SEQ ID NO 13] and to have coordinates 2701-2740 relative to the CACNA1I mRNA (AF393329).

The 5′ “forward” ORF3 primer is designed to have the nucleotide sequence of 5′ CTCTCACTGCAGCCGGACCCCATGCTGGTGGCCCTGGGCT 3′ [SEQ ID NO 14] and to have coordinates 2701-2740 relative to the CACNA1I mRNA (AF393329). The 3′ “reverse” ORF3 primer is designed to have the nucleotide sequence of 5′ TTGGTCACAGGCTGCTGGTCCACAGCAACAGCA TCCAGTC 3′ [SEQ ID NO 15] and to have coordinates 4061-4100 relative to the CACNA1I mRNA (AF393329).

The 5′ “forward” ORF4 primer is designed to have the nucleotide sequence of 5′ GACTGGATGCTGTTGCTGTGGACCAGCAGCCTGTGACCAA 3′ [SEQ ID NO 16] and to have coordinates 4061-4100 relative to the CACNA1I mRNA (AF393329). The 3′ “reverse” ORF4 primer is designed to have the nucleotide sequence of 5′ CTAGAAGGCACAGTCGAGGCTGATCAGCGGGTTTAA ACTCACTACTAGGCGGACGGGGACGGGGTGGGG 3′ [SEQ ID NO 17].

The above-described primer sequences, primer coordinates relative to CACNA1I mRNA (AF393329), and amplicon sizes are listed in Table 1. The primer sequences in italics are “tails” that are incorporated into the PCR amplicons and facilitate subsequent plasmid recombination events in yeast.

TABLE 1
PCR primers used to amplify CACNAII ORF1-4 segments

| | | | AF393329 | Amplicon |
|---|---|---|---|---|
| Primer name | SEQ ID NO | Primer sequence | coordinates | size |
| ORF1 forward | SEQ ID NO 10 | ATCGAAATTAATACGACTCACTATAGGGAGACCCAAG | 1-25 | 1390 bp |
| | | CTGATGGCTGAGAGCGCCTCCCCGCCCT | | |
| ORF1 reverse | SEQ ID NO 11 | CTTGGCCTTGCGCAGGATGTGGCAGACATACTGGAAG | 1311-1350 | 1390 bp |
| | | ATC | | |
| ORF2 forward | SEQ ID NO 12 | GATCTTCCAGTATGTCTGCCACATCCTGCGCAAGGCC | 1311-1350 | 1430 bp |
| | | AAG | | |
| ORF2 reverse | SEQ ID NO 13 | AGCCCAGGGCCACCAGCATGGGGTCCGGCTGCAGTGA | 2701-2740 | 1430 bp |
| | | GAG | | |
| ORF3 forward | SEQ ID NO 14 | CTCTCACTGCAGCCGGACCCCATGCTGGTGGCCCTGG | 2701-2740 | 1400 bp |
| | | GCT | | |
| ORF3 reverse | SEQ ID NO 15 | TTGGTCACAGGCTGCTGGTCCACAGCAACAGCATCCA | 4061-4100 | 1400 bp |
| | | GTC | | |
| ORF4 forward | SEQ ID NO 16 | GACTGGATGCTGTTGCTGTGGACCAGCAGCCTGTGAC | 4061-4100 | 1664 bp |
| | | CAA | | |
| ORF4 reverse | SEQ ID NO 17 | CTAGAAGGCACAGTCGAGGCTGATCAGCGGGTTTAAA | No match | 1664 bp |
| | | CTCACTACTAGGCGGACGGGGACGGGGTGGGG | | |

RT-PCR

Four segments of CACNA1I cDNA sequence corresponding to ORF1, ORF2, ORF3, and ORF4 are cloned using a combination of reverse transcription (RT) and polymerase chain reaction (PCR). More specifically, about 25 ng of whole brain total RNA (BD Biosciences Clontech, Palo Alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTP 1 μl of enzyme from the Clontech (Palo Alto, Calif.) Advantage 2 PCR kit. PCR is done in a Gene Amp PCR System 9700 (Applied Biosystems, Foster City, Calif.) using the CACNA1I ORF1, ORF2, ORF3, or ORF4 “forward” and “reverse” primers [SEQ ID NOs 10-17]. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are follow a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR had yielded products of the expected size, in the case of the predicted ORF1 mRNA, a product of about 1,390 base pairs, in the case of the predicted ORF2 mRNA, a product of about 1,430 base pairs, in the case of ORF3 mRNA, a product of about 1,400 base pairs, and in the case of ORF4, a product of about 1,664 base pairs. The remainder of the 50 μl PCR reactions from whole brain is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified ORF1, ORF2, ORF3, and ORF4 RT-PCR product from adult whole brain are cloned into an Invitrogen pCR2.1 vector using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2×LB medium. These liquid cultures are incubated overnight on a roller at 37° C. Plasmid DNA is extracted from these cultures using the Qiagen (Valencia, Calif.) Qiaquik Spin Miniprep kit.

Four putative ORF1, ORF2, ORF3, and ORF4 clones are identified and prepared for a PCR reaction to confirm the presence of the expected ORF1, ORF2, ORF3, and ORF4 structure. A 25 μl PCR reaction is performed as described above (RT-PCR section) to detect the presence of ORF1, ORF2, ORF3, and ORF4, except that the reaction includes miniprep DNA from the TOPO TA/ORF1, TOPO TA/ORF2, TOPO TA/ORF3 and TOPO TA/ORF4 ligation, respectively, as a template. About 10 μl of each 25 μl PCR reaction is run on a 1% Agarose gel and the DNA bands generated by the PCR reaction are visualized and photographed on a UV light box to determine which miniprep samples have PCR product of the size predicted for the corresponding ORF1, ORF2, ORF3, and ORF4 mRNA. Clones having the ORF1 , ORF2, ORF3, and ORF4 structure are identified based upon amplification of an amplicon band of 1390, 1430, 1400, and 1664 base pairs, respectively. DNA sequence analysis of the ORF1 , ORF2, ORF3, and ORF4 cloned DNA confirms a polynucleotide sequence representing the ORF1 , ORF2, ORF3, and ORF4 based upon CACNA1I mRNA (AF393329).

The TOPO TA/ORF1 , TOPO TA/ORF2, TOPO TA/ORF3 and TOPO TA/ORF4 ligations are used as template for a brief (≦10 cycle) PCR as described above (RT-PCR section) using ORF1 primers [SEQ ID NOs 10 and 11], ORF2 primers [SEQ ID NOs 12 and 13], ORF3 primers [SEQ ID NOs 14 and 15]and ORF4 primers [SEQ ID NOs 16 and 17], respectively, to create sufficient quantities of the amplicons for recombination.

Construction of Cycloheximide-Resistant Saccharomyces cerevisiae Strain

A cycloheximide-based counterselection is used to increase specificity of cloning by homologous recombination relative to nonspecific vector background (Raymond et al, 2002, Genome Res. 12:190-197). Plasmid re-circularization is the primary source of background in recombinational cloning experiments (Boulton and Jackson, 1996, Nucleic Acids Res. 24:4639-4648; Raymond et al., 1999, Biotechniques 26:134-8, 140-1). The yeast strain used in this study, CMY1-5, is cycloheximide resistant (CYH2^{R}). The introduction of the wild-type CYH2 allele into this yeast strain confers dominant sensitivity to cycloheximide. The position of the CYH2 gene in the cloning vector relative to the targeted recombination sites is designed such that the CYH2 gene is lost in recombinant clones but retained in most end-joined plasmids. Therefore, yeast cells containing recombinant plasmids are selected in the presence of cycloheximide, while yeast cells containing non-recombinant plasmids are sensitive to the drug. However, any marker that confers dominant sensitivity can be used in a counterselection experiment, such as URA3, 5-fluoroorotic acid, LYS2 and α-amino adipic acid, or CAN1.

A cycloheximide resistant strain is generated from the cycloheximide sensitive yeast strain BY4709 (Brachmann et al, 1998, Yeast 14:155-132). Two overlapping 1 kb products from the CYH2 gene are amplified from BY4709 yeast strain using two sets of tailed primers (hereafter Set 1 and Set 2) listed in Table 2, which introduce a critical Q→E amino acid change at residue 38 in the CYH2 protein, thereby conferring drug resistance. The 5′ “forward” Set 1 primer is designed to have the nucleotide sequence of 5′ ATACGACTCACTATAGGGAGACCCAAGCTGGCTAGTTAAGTATG TTTATATATGGATTTTGAAA 3′ [SEQ ID NO 18]. The 3 ′ “reverse” Set 1 primer is designed to have the nucleotide sequence of 5′ GTA GAGGTATGGCCGGTGGTGAACATCACCACAGAATTAAC 3′ [SEQ ID NO 19]. The 5′ “forward” Set 2 primer is designed to have the nucleotide sequence of 5′ TCCACCACACTGGACTAGTGGATCCGAGCTCGGTACCAAGGCCCGGGCATGCTACGTACC TGTTTAACTCTTC 3′ [SEQ ID NO 20]. The 3′ “reverse” Set 2 primer is designed to have the nucleotide sequence of 5′ GTTAATTCTGTGGTGATGTTCACCACCGGCCATACCTCTAC 3′ [SEQ ID NO 21].

TABLE 2

Tailed PCR primer sets used to amplify CYH2
and introduce a drug-resistant mutation

| Primer | | |
|---|---|---|
| Name | SEQ ID NO | Primer Sequence |
| Set 1 | SEQ ID NO 18 | ATACGACTCACTATAGGGAGACCCAAGCT |
| Forward | | GGCTAGTTAAGTATGTTTATATATGGATT |
| | | TTGAAA |
| Set 1 | SEQ ID NO 19 | GTAGAGGTATGGCCGGTGGTGAACATCAC |
| Reverse | | CACAGAATTAAC |
| Set 2 | SEQ ID NO 20 | TCCACCACACTGGACTAGTGGATCCGAGC |
| Forward | | TCGGTACCAAGGCCCGGGCATGCTACGTA |
| | | CCTGTTTAACTCTFC |
| Set 2 | SEQ ID NO 21 | GTTAATTCTGTGGTGATGTTCACCACCGG |
| Reverse | | CCATACCTCTAC |

Recombination of the Set 1 and Set 2 CYH2 amplicons into the endogenous yeast CYH2 gene confers cycloheximide resistance. 1 μg each of the Set 1 and Set 2 PCR amplicons, which incorporate the Q38E mutation, are cotransformed by electroporation as described in Raymond et al. (2002, Genome Res. 12:190-197) with yeast strain BY4709 (Brachmann et al., 1998 Yeast, 14:115-132), and cycloheximide resistant colonies are selected on media containing 1 μg/ml cycloheximide (Sigma, St. Louis, Mo.). Yeast transformation methods, including electroporation, lithium acetate treatment, and spheroplasting, are also discussed in Gietz and Woods (2001 Biotechniques, 30:816-820, 822-826, 828). One cycloheximide resistant strain, CMY1-5 (Matα, ura3Δ, cyh2^{R}) is used for all subsequent studies.

Construction of Yeast Plasmids

Plasmids that are assembled in yeast by recombination typically include sequence elements that allow their selection in yeast (e.g., CYH2 or URA3 resistance). Sequence elements that permit plasmid replication include a yeast centromere sequence and yeast DNA autonomously replicating sequence. DNA sequences for selection (e.g., ampicillin, kanamycin, or chloramphenicol resistance) and replication (e.g., colE1 or mini F′) in Escherichia coli are typically included in the plasmid to allow transformation of E. coli for the preparation of large quantities of recombinant plasmid. Isolation of yeast plasmid for bacterial transformation is described in Hoffman and Winston (1987, Gene 57:267-72). One such embodiment of a yeast plasmid is the sequences found in the vector pRS316 (Sikorski and Hieter, 1989, Genetics 122:19-27). Additional elements including promoters, terminators, and selectable markers for recombinant expression in mammalian cells can be found in commercially available plasmid vectors and incorporated into yeast cloning vectors.

Overlapping sequences are used to target recombinational cloning of DNA fragments into specific sites in the target yeast plasmid. The region of overlap can be as short as 20 bp, but is optimally 40 bp or longer (Hudson et al, 1997, Genome Res. 7:1169-1173; Oldenburg et al., 1997 Nucleic Acids Res. 25:451-452; Hua et al., 1997, Plasmid 38:91-96; Raymond et al, 1999, Biotechniques, 26:134-8, 140-1). Sequence homology for these overlapping regions may be provided by amplification of target sequences with PCR primers that include varying lengths of base pair extensions (“tails”) that become incorporated into the amplicons. Alternatively, synthetic oligonucleotide recombination linkers that provide sequence homology at each end to the two unrelated DNA molecules to be joined may be used (Raymond et al, 2002, Genome Res. 12:190-197; DeMarini et al., 2001, Biotechniques 30:520-523).

To create the cloning vectors used for this study, plasmid pRS416 (ATCC No. 87521) (Sikorski and Hieter, 1989, Genetics, 122:19-27) is digested with SspI; and plasmid pENTR11 (InVitrogen, Carlsbad, Calif.) is digested with NheI. BY4709 yeast strain is cotransformed with the two plasmid vector fragments and the recombinational linkers listed in Table 3 [SEQ ID NOs 22-25] by electroporation (Raymond et al., 2002 Genome Res. 12:190-197) to form the resulting pCMR2 plasmid [SEQ ID NO 26]. All subsequent transformation steps for plasmid construction are performed with yeast strain BY4709 using the referenced electroporation method unless otherwise indicated.

TABLE 3

Linkers used to joining
pRS416 (SspI) and pENTR11 (NheI).

| | |
|---|---|
| SEQ ID NO | Linker Sequence |

| | |
|---|---|
| SEQ ID NO 22 | TGCGTTATCCCCTGATTCTGTGGATAACCGTATTACC |
| | GCTGGGTAATAACTGATATAATTAAATTGAAGCTCTA |
| | ATTTGT |
| SEQ ID NO 23 | ACAAATTAGAGCTTCAATTTAATTATATCAGTTATTA |
| | CCCAGCGGTAATACGGTTATCCACAGAATCAGGGGAT |
| | AACGCA |
| SEQ ID NO 24 | AATTTAAATTATAATTATTTTTATAGCACGTGATGAA |
| | AAGAGCATGGATCTCGGGGACGTCTAACTACTAAGCG |
| | AGAGTA |
| SEQ ID NO 25 | TACTCTCGCTTAGTAGTTAGACGTCCCCGAGATCCAT |
| | GCTCTTTTCATCACGTGCTATAAAAATAATTATAATT |
| | TAAATT |

pCMR2 plasmid [SEQ ID NO 26] is cut with Smal and then recombined with the linkers in Table 4 [SEQ ID NO 27, 28] to produce pCMR3 [SEQ ID NO 29].

TABLE 4
Linkers used to convert pCMR2 to pCMR3.

| | |
|---|---|
| SEQ ID NO | Linker Sequence |

| | |
|---|---|
| SEQ ID NO 27 | ACTTTGTACAAAAAAGCAGGCTTCGAAGGAGATAGAA |
| | CCAGCCCGGGCGCCGCACTCGAGATATCTAGACCCAG |
| | CTTTCTTGTACAAA |
| SEQ ID NO 28 | TTTGTACAAGAAAGCTGGGTCTAGATATCTCGAGTGC |
| | GGCGCCCGGGCTGGTTCTATCTCCTTCGAAGCCTGCT |
| | TTTTTGTACAAAGT |

pCMR3 [SEQ ID NO 29] is digested with SspI, and pCCFOS1 (EpiCentre Technologies, Madison, Wis.) is cut with Sa1I. The vector fragments are then joined with the linkers shown in Table 5 [SEQ ID NOs 30-33], resulting in plasmid pCMR7 [SEQ ID NO 34].

TABLE 5
Linkers used to join pCMR3 and pCCFOS1.

| | |
|---|---|
| SEQ ID NO | Linker Sequence |

| | |
|---|---|
| SEQ ID NO 30 | GTTAACCGGGCTGCATCCGATGCAAGTGTGTCGCTGT |
| | CGAGGGTAATAACTGATATAATTAAATTGAAGCTCTA |
| | ATTTGT |
| SEQ ID NO 31 | ACAAATTAGAGCTTCAATTTAATTATATCAGTTATTA |
| | CCCTCGACAGCGACACACTTGCATCGGATGCAGCCCG |
| | GTTAAC |
| SEQ ID NO 32 | ATAAAATCATTATTTGCCATCCAGCTGCAGCTCTGGC |
| | CCGTCGAATTTCTGCCATTCATCCGCTTATTATCACT |
| | TATTCA |
| SEQ ID NO 33 | TGAATAAGTGATAATAAGCGGATGAATGGCAGAAATT |
| | CGACGGGCCAGAGCTGCAGCTGGATGGCAAATAATGA |
| | TTTTAT |

Plasmid pCMR7 [SEQ ID NO 34] is cut with SrfI; plasmid pcDNA3.1 mycHIS A (InVitrogen) is cut with SspI. The resulting plasmid fragments are joined with the linkers shown in Table 6 [SEQ ID NOs 35-38], yielding plasmid pCMR9 [SEQ ID NO 39].

TABLE 6

Linkers used to join pCMR7
and pcDNA3.1 mycHIS A.

| | |
|---|---|
| SEQ ID NO | Linker Sequence |

| | |
|---|---|
| SEQ ID NO 35 | ACGGTTATCCACAGAATCAGGGGATAACGCAGGAAAG |
| | AACATAAAATCATTATTTGCCATCCAGCTGCAGCTCT |
| | GGCCCG |
| SEQ ID NO 36 | CGGGCCAGAGCTGCAGCTGGATGGCAAATAATGATTT |
| | TATGTTCTTTCCTGCGTTATCCCCTGATTCTGTGGAT |
| | AACCGT |
| SEQ ID NO 37 | AATTTAAATTATAATTATTTTTATAGCACGTGATGAA |
| | AAGTCCGCGCACATTTCCCCGAAAAGTGCCACCTGAC |
| | GTCGAC |
| SEQ ID NO 38 | GTCGACGTCAGGTGGCACTTTTCGGGGAAATGTGCGC |
| | GGACTTTTCATCACGTGCTATAAAAATAATTATAATT |
| | TAAATT |

Construct pCMR9 [SEQ ID NO 39] is cut with HindIII. The yeast CYH2 gene is amplified as two 1 kb overlapping pieces from yeast strain BY4709 (Brachmann et al, 1998, Yeast 14:155-132), using the two sets of tailed primers listed in Table 7. The sequences of one set of CYH2 primers are set forth in SEQ ID NOs 18 and 40; the sequences of the second set of primers are set forth in SEQ ID NOs 20 and 41. The full length CYH2 gene is assembled into pCMR9 by cotransformation of CMY1-5 yeast strain to produce pCMR11 [SEQ ID NO 42] (see Table 8).

TABLE 7

Tailed PCR primers used to amplify CYH32
for recombination into pCMR9.

| | |
|---|---|
| SEQ ID NO | Primer Sequence |

| | |
|---|---|
| SEQ ID NO 18 | ATACGACTCACTATAGGGAGACCCAAGCTGGCTAGTT |
| | AAGTATGTTTATATATGGATTTTGAAA |
| SEQ ID NO 40 | GTAGAGGTATGGCCGGTGGTCAACATCACCACAGAAT |
| | TAAC |
| SEQ ID NO 20 | TCCACCACACTGGACTAGTGGATCCGAGCTCGGTACC |
| | AAGGCCCGGGCATGCTACGTACCTGTTTAACTCTTC |
| SEQ ID NO 41 | GTTAATTCTGTGGTGATGTTGACCACCGGCCATACCT |
| | CTAC |

TABLE 8
Composition of pCMR10 and CMR11 plasmids

| Nucleotide coordinates | Functional description of sequence |
|---|---|
| 1-6013 | Copy-control ™ E. coli origin of replication from pCC1FOS (Epicentre Technologies, |
| | Madison, WI). |
| 6014-7884 | Yeast URA3 gene, ARS4 autonomously replicating sequence and CEN6 centromere |
| | from pRS316 (Sikorski and Helter, 1989). |
| 7885-8825 | Mammalian CMV promoter from InVitrogen (Carlsbad, CA) vector pcDNA3.1/myc- |
| | HIS A. |
| 8826-10,774 | Yeast CYH2 gene amplified from strain BY4709 (Brachmann et al. 1998) |
| 10,775-10,782 | Engineered SrfI restriction site. |
| 10,783-13,556 | Mammalian poly-adenylation sites, selectable markers, SV40 origin, etc. from |
| | pcDNA3.1/myc-HIS A. |
| 13,557-13,596 | DNA sequence from InVitrogen vector pENTR11. |
| 13,597-14,567 | pCMR10 - specific; kanamycin resistance gene from pENTR11. |
| 13,597-14,561 | pCMR11 - specific; chloramphenicol resistance gene from pCC1FOS. |

Plasmid pCMR11 [SEQ ID NO 42] carries a chloramphenicol resistance gene. To convert this resistance gene to a kanamycin resistance marker, pCMR11 [SEQ ID NO 42] is digested with BspEI. The kanamycin resistance gene is amplified from pENTR11 (InVitrogen) with the tailed primers shown in Table 9 [SEQ ID NOs 43 and 44]. The digested pCMR 11 [SEQ ID NO 42] and kanamycin amplicon are joined by recombination in yeast strain CMY1-5. The resulting plasmid is called pCMR10 [SEQ ID NO 45] (see Table 8).

TABLE 9

Tailed PCR primers used to amplify the
kanamycin resistance marker from pENTR11
for recombinationinto pCMR1 1.

| | |
|---|---|
| SEQ ID NO | Primer sequence |

| | |
|---|---|
| SEQ ID NO 43 | ATAAAATCATTATTTGCCATCCAGCTGCAGCTCTGGC |
| | CCGTGTCTCAAAATCTCTGATGTTACAT |
| SEQ ID NO 44 | TTTCTCTGTCCTTCCTGTGCGACGGTTACGCCGCTCC |
| | ATGGTCTGACGCTCAGTGGAACGGGGCC |

Assembly of CACNA1Isv1 Full-Length Clone and Yeast Transformation

In addition to sequence homology between the two sequences to be joined, the quantities of acceptor vector and donor PCR fragments are critical for efficient recombination. As described in Raymond et al. (2002, Genome Res. 12:190-197), 100 ng of acceptor vector and 1 μg of DNA donor fragment are used to transform yeast cells. Assembly of the full-length CACNA1Isv1 full length clone by homologous recombination between overlapping pieces of ORF1, ORF2, ORF3, ORF4, and the expression vector is performed by simultaneous transformation of these pieces into yeast cells. All yeast transformation steps described in subsequent paragraphs are performed by electroporation (Raymond et al., 2002 Genome Res. 12:190-197). Yeast transformation methods (electroporation, lithium acetate treatment, or spheroplasting) are also compared by Gietz and Woods (2001, Biotechniques 30:816-820, 822-826, 828).

To construct the CACNA1Isv1 clone, 1 μg each of ORF1, ORF2, ORF3, and ORF4 fragments that have undergone a 10-cycle PCR as described previously, and 100 ng of SrfI-digested pCMR11 are used to cotransform 100 μl of CMY1-5 yeast strain (Matα, URA3Δ, CYH2^{R}). The overlapping DNA between the sequential DNA fragments dictates that most yeast transformants will possess the correctly assembled construct. Ura+, cycloheximide resistant colonies are selected for subsequent preparation and transformation of E. coli. Plasmid DNA extracted from E. coli is analyzed by restriction digest to confirm the presence of the alternative splicing of exon 15 to exon 17 in the CACNA1Isv1 clone. Eight CACNA1Isv1 clones are sequenced to confirm identity, and the clones with sequence corresponding to CACNA1Isv1 are used for protein expression in multiple systems, including mammalian cells.

The polynucleotide sequence of CACNA1Isv1 mRNA [SEQ ID NO 3] possesses a unique 276 base pair region corresponding to intron 32 of the CACNA1I gene. Addition of the 276 base pair region does not alter the protein translation reading frame. However, the 276 base pair region does introduce a stop codon 3 nucleotides before the intron 32-exon 33 splice junction. Therefore, in addition to possessing 91 unique amino acids corresponding to intron 32, the CACNA1Isv1 polypeptide is also lacking a C-terminal 425 amino acid region corresponding to exons 33-37 of the full length coding sequence of the reference CACNA1I mRNA (AF393329).

### Example 3

### Real-Time Quantitative PCR/TAQman

To determine the relative mRNA abundance of CACNA1Isv1 alternatively spliced isoform to the CACNA1I reference protein (AAM67414) and the previously identified Δexon 33 isoform (Mittman et al., 1999, Neurosci. Lett. 269:121-124), a real-time quantitative PCR assay was used. Materials and methods for quantification of splice variants using real-time PCR, using boundary specific probes are known in the art (Kafert et al., 1999 Anal. Biochem. 269:210-213; Vandenbroucke et al, 2001 Nucleic Acids Res. 29:E68-8; Taveau et al., 2002 Anal. Biochem. 305:227-235).

Reverse Transcription

RNA samples from a panel of human tissues including fetal brain, hypothalamus, nucleus basilis, basal ganglia, thalamus, temporal cortex, cerebellum, hippocampus, medulla oblongata, nucleus accumbens, pons, and putamen (ClonTech, Palo Alto, Calif.), were reverse transcribed using the Applied Biosystems (Foster City, Calif.) TAQman reverse transcription kit N808-0234 following manufacturer's instructions. A 50 μl reaction contained:

| | |
|---|---|
| 5 | μl 10X RT buffer |
| 11 | μl MgCl₂ solution |
| 10 | μl dNTP solution |
| 2.5 | μl random hexamer primer |
| 1 | μl RNAse OUT |
| 3 | μl Multiscribe reverse transcriptase |
| 1 | μg of RNA |

H₂O to a final volume of 50 μl.

To convert RNA to single-stranded cDNA, the reaction mixture was incubated at the following conditions: 25° C. for 10 minutes, 37° C. for 60 minutes, 95° C. for 5 minutes. Th was then placed on ice prior to use.

Plasmid Construction and Standard Curve

Plasmids carrying the reference CACNA1I sequence, Δexon 33 isoform, and alternatively spliced isoform CACNA1Isv1 were constructed in order to prepare a standard curve. The CACNA1I cDNA region spanning nucleotides from exon 32 to exon 34 was amplified with exon 32 primer 5′ CTTGTGCCGGCGCTGCTACTCGCCTG 3′ [SEQ ID NO 8] and exon 34 primer 5′ AGGATGGACGAGGACCTGTCTGAGTTC 3′ [SEQ ID NO 9] from brain cDNA. The PCR products were cloned into pCR2.1 vector (Invitrogen). The cloning reaction was used to transform TOP10 chemically competent E. coli cells, and plasmid DNA was extracted using the Qiagen (Valencia, Calif.) Qiaquick Spin Miniprep kit. DNA was quantified using a UV spectrometer. Sequence identities of plasmid clones containing the CACNA1I reference sequence; the Δexon 33 isoform, which lacks 39 nucleotides at the 5′ end of exon 33; and the alternatively spliced CACNA1Isv1 sequence, which retains intron 32, were verified.

To construct a standard curve with the plasmid clones carrying the CACNA1I reference sequence, Δexon 33 sequence, and CACNA1Isv1 sequence, ten-fold serial dilutions of the plasmids were used to obtain a range of five orders of magnitude. Final plasmid concentrations of 100 pg, 10 pg, 1 pg, 0.1 pg, and 0.01 pg were amplified using real-time PCR. Fluorescence emission values were plotted onto a standard curve, permitting quantification of the experimental samples compared to the standard curve.

Real-Time PCR

TAQman primers and probes used to quantify the CACNA1Isv1 isoform were designed and synthesized as pre-set mixtures (Applied Biosystems, Foster City, Calif.). The sequences of the TAQman primers and probes used to quantify the CACNA1I reference form [SEQ ID NOs 46, 47, and 48] and CACNA1Isv1 isoform [SEQ ID NOs 49, 50, and 51] and the previously identified Δexon 33 CACNA1I isoform [SEQ ID NOs 52, 53, and 54] (Mittman et al., 1999, Neurosci. Lett. 269:121-124) are shown in Table 10. Splice junction specific probes were labeled with the 6-FAM fluorphore at the 5′ end (FAM) and a non-fluorescent quencher at the 3′ end (NFQ). Real-time PCR was performed on a panel of cDNAs including human fetal brain, hypothalamus, nucleus basilis, basal ganglia, thalamus, temporal cortex, cerebellum, hippocampus, medulla oblongata, nucleus accumbens, pons, and putamen, using the TaqMan Universal PCR Master Mix (Applied Biosystems, Foster City, Calif.). The TAQman reaction contained:

| 96-well format | 384-well format | |
|---|---|---|
| 12.5 μl | 5 μl | TAQman Universal MasterMix |
| 1.25 μl | 0.5 μl | Primer-probe mix |
| 6.25 μl | 2.5 μl | H₂O |
| 5 μl | 2 μl | cDNA or plasmid DNA. |

TABLE 10
Primers and probes used to quantify CACNA1I isoforms.

| | | | |
|---|---|---|---|
| Name | SEQ ID NO | Sequence | Specificity |

| | | | |
|---|---|---|---|
| CACNA1I reference | SEQ ID NO 46 | GCCGGCGCTGCTACT | CACNA1I reference |
| forward primer | | | |
| CACNA1I reference | SEQ ID NO 47 | GACGCTGTCCAGCCACA | CACNA1I reference |
| reverse primer | | | |
| CACNA1I reference | SEQ ID NO 48 | FAM-CTGCCCAGGAGAACC-NFQ | CACNA1I reference |
| probe | | | |
| CACNA1Isv1 | SEQ ID NO 49 | GCTGATAATCCCGCCTGTCC | CACNA1Isv1 |
| forward primer | | | |
| CACNA1Isv1 | SEQ ID NO 50 | TGATGATTAAAGAGACGCTGTCCAG | CACNA1Isv1 |
| reverse primer | | | |
| CACNA1Isv1 probe | SEQ ID NO 51 | FAM-CGCCTAGGAGAACCTGTGG-NFQ | CACNA1Isv1 |
| CACNA1I Δexon 33 | SEQ ID NO 52 | GCCGGCGCTGCTACT | CACNA1I Δexon 33 |
| forward primer | | | |
| CACNA1I Δexon 33 | SEQ ID NO 53 | GGTCAGCTCCCCCTCCAA | CACNA1I Δexon 33 |
| reverse primer | | | |
| CACNA1I Δexon 33 | SEQ ID NO 54 | FAM-CTGCCCAGGACTCC-NFQ | CAGNA1I Δexon 33 |
| probe | | | |

The TAQman reactions were performed on an ABI Prism 7900HT Sequence Detection System (Applied Biosystems, Foster City, Calif.). The thermocycling conditions were 50° C. for 2 minutes, 95° C. for 10 minutes, and 40 cycles of 95° C. for 15 seconds and 60° C. for 1 minute. Data fluorescence emission was performed by the Sequence Detector Software (SDS) (Applied Biosystems, Foster City, Calif.). Briefly, an amplification plot was generated for each sample, which showed cycle number on the x-axis vs. ΔRₙ on the y-axis. Rₙ is the fluorescence emission intensity of the reporter dye normalized to a passive reference, and ΔRₙ is the Rₙ value of the reaction minus the Rₙ of an un-reacted sample. A threshold cycle (C_{T}) value, the cycle at which a statistically significant increase in ΔRₙ is first detected, was calculated from the amplification plot. The threshold was automatically calculated by the SDS as the 10-fold standard deviation of the Rₙ in the first 15 cycles. The obtained C_{T} values were exported Microsoft Excel for analysis as recommended by the manufacturer (Applied Biosystems, Foster City, Calif.). Standard curve plots showing the log₁₀ [input cDNA] vs. C_{T} values were constructed. Referring to the standard curve, C_{T} values for the experimental samples were then used to calculate the input amount of the CACNA1I isoform cDNA. The quantity of each isoform was then divided by the sum of all of the mutually exclusive splicing events in each tissue (intron 32 retention+reference CACNA1I+Δexon 33). The quantities of each isoform were presented as a fraction of the total expression of all the isoforms for that particular gene region, in each tissue. Quantitative analysis of the real-time PCR data indicated that the CACNA1Isv1 isoform was present in all the tissues assayed, but was the least abundant isoform. For example, the abundance of the CACNA1Isv1 isoform was measured at 15.7% compared to the reference CACNA1I (49.2%) and Δexon 33 isoform (35.1%) in the cerebellum.

### Example 4

### Analyzing Ion Channel Activity of CACNA1I and Other Calcium Channel Isoforms

To express CACNA1I and other calcium channel isoform channels in Xenopus oocytes, cDNA encoding the appropriate channel is cloned into pCMR11 [SEQ ID NO 42] by recombination in yeast and subcloned into standard expression vectors, such as pGEM-HEA. pGEM-HEA vector was initially created from pGEM-HE and contains 5′ and 3′ untranslated regions from a Xenopus β-globin gene, resulting in high levels of expression (Liman et al., 1992, Neuron 9:861-871). Plasmids are linearized, and RNA is transcribed using the T7 (or SP6) RNA polymerase and the mMessage mMachine kit from Ambion (Austin, Tex.). Each oocyte is injected with 10 ng of cRNA in a volume of 50 nl. Oocytes are then incubated for 3-5 days at 18° C. in ND-96 medium (96 mM NaCl, 2 mM, KCl, 1 mM MgCl₂, 1.8 mM CaCl₂, 5 mM HEPES, pH 7.5) supplemented with 0.1% gentamicin (Sigma). Currents are recorded at room temperature with a conventional two-electrode voltage clamp (GeneClamp500, Axon Instruments, Foster City, Calif.) using standard methods previously described and known in the art (Dascal, 1987, CRC Crit. Rev. Biochem. 22:317-387). The microelectrodes are filled with 3M KCl, which have resistances of 1-2MΩ. Cells are continuously perfused with ND-96 at 2-5 ml/min at room temperature. Membrane voltage is clamped at −100 mV, and a current-voltage relationship is measured to determine the voltage that produced the maximum inward current. Typically, oocytes are depolarized to −30 or −40 mV. Data is typically filtered at 1-2 kHz and digitized at 10-20 kHz using an Axopatch 200B amplifier, a Digidata 1200 interface, and pClamp6 software (Axon Instruments). Leak current is subtracted using the scaled current observed with a P/n protocol (Benzanilla and Armstrong, 1977 J. Gen. Physiol. 70:549-566). The endogenous oocyte Ca²⁺-activated Cl⁻ current is inhibited prior to recording with an injection of 2-5 mM final concentration of BAPTA. After a stable peak current is obtained, the indicated concentration of venom, toxin, or compound is added by perfusing the recording chamber with ND-96 containing the sample, typically at 2-5 ml/min until stable block is achieved. The oocyte is subsequently perfused with fresh ND-96 to assess reversibility of inhibition.

Cell Culture and HEK Cell Electrophysiology:

For whole-cell voltage-clamp recordings, stably transfected HEK-293 cells or other eukaryotic cell lines expressing CACNA1I or other calcium channel isoforms are used. Retro-virus vectors may be used to generate cell lines expressing CACNA1I or other calcium channel isoform cell lines. The CACNA1I or other calcium channel isoform is cloned in a retroviral expression vector (pLCNX, Clontech). Subsequently virus particles are used to infect HEK-293 cells, and a cell line stably expressing the CACNA1I channel is selected. Cells are maintained in DMEM (Dulbecco's modified Eagles medium) supplemented with 10% fetal bovine serum (FBS), penicillin (100 U/ml), and streptomycin (100 μg/ml). Cells are plated on poly-D-lysine coated cover slips 1-3 days prior to recording and are then washed with the external solution immediately prior to recording to remove any endogenous redox agents in the FBS.

Single cell recordings of currents through voltage-activated CACNA1I or other calcium channel isoforms are performed at room temperature (20-22° C.) using the whole cell patch-clamp technique (Hamill et al., 1981, Pflugers Archives 391: 85-100). Currents are recorded using Axopatch 200B patch clamp amplifier. Data are stored on a personal computer equipped with pClamp6 software. Patch pipets are made from borosilicate glass tubing (World Precision Instruments, Sarasota, Fla.), fire-polished, and coated with Sylgard and have a resistance of 1-3MΩ when filled with an internal solution (110 mMCsCl, 10 mM EGTA, 10 mM HEPES, 3 mM Mg-ATP, 0.6 mM GTP, pH 7.2 with CsOH) measured in the recording medium. External solution contains 2 mM CaCl2, 160 mM TEA-Cl, 10 mM HEPES 9 (pH 7.4 with TEA-OH). Recordings are filtered at 5 kHz. Series resistance and capacitance are compensated using the whole-cell parameters of the Axopatch 200B amplifier. Leak and residual capacitive currents are subtracted using a P/n procedure. Data are analyzed using pClamp6, Excel (Microsoft), and Prism (GraphPad) and are given as mean±standard error mean.

Data are collected using standard pulse protocols and are analyzed to measure calcium current properties that include voltage-dependence, steady- state characteristics, kinetics, and re-priming. Methods of electrophysiological measurements and analysis have been previously described and are known in the art (Lee et al., 1999, J. Neurosci. 19:1912-1921; Chemin et al., 2001, EMBO J. 20:7033-7040; Chemin et al., 2001, Biophys. J. 80:1238-1250; Chemin et al., 2001, Eur. J. Neurosci. 14:1678-1686). These measurements are carried out both in control cells expressing CACNA1I or other calcium channel isoforms, and in cells expressing CACNA1I or other calcium channel isoforms that also have been exposed to the compound to be tested.

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

What is claimed:

1. A purified human nucleic acid comprising SEQ ID NO 3, or the complement thereof.

2. The purified nucleic acid of claim 1, wherein said nucleic acid comprises a sequence encoding SEQ ID NO 4.

3. The purified nucleic acid of claim 1, wherein said nucleic acid encodes a polypeptide consisting of SEQ ID NO 4.

4. A purified polypeptide comprising SEQ ID NO 4.

5. The polypeptide of claim 4, wherein said polypeptide consists of SEQ ID NO 4.

6. An expression vector comprising a nucleotide sequence encoding SEQ ID NO 4, wherein said nucleotide sequence is transcriptionally coupled to an exogenous promoter.

7. The expression vector of claim 6, wherein said nucleotide sequence encodes a polypeptide consisting of SEQ ID NO 4.

8. The expression vector of claim 6, wherein said nucleotide sequence comprises SEQ ID NO 3.

9. The expression vector of claim 6, wherein said nucleotide sequence consists of SEQ ID NO 3.

10. A method of screening for compounds able to bind selectively to CACNA1Isv1 comprising the steps of:
(a) providing a CACNA1Isv1 polypeptide comprising SEQ ID NO 4;
(b) providing one or more calcium channel isoform polypeptides that are not CACNA1Isv1;
(c) contacting said CACNA1Isv1 polypeptide and said calcium channel isoform polypeptide that is not CACNA1Isv1 with a test preparation comprising one or more compounds; and
(d) determining the binding of said test preparation to said CACNA1Isv1 polypeptide and to said calcium channel isoform polypeptide that is not CACNA1Isv1, wherein a test preparation which binds to said CACNA1Isv1 polypeptide, but does not bind to said calcium channel isoform polypeptide that is not CACNA1Isv1, contains a compound that selectively binds said CACNA1Isv1 polypeptide.

11. The method of claim 10, wherein said CACNA1Isv1 polypeptide is obtained by expression of said polypeptide from an expression vector comprising a polynucleotide encoding SEQ ID NO4.

12. The method of claim 1, wherein said polypeptide consists of SEQ ID NO 4.

13. A method for screening for a compound able to bind to or interact with a CACNA1Isv1 protein or a fragment thereof comprising the steps of:
(a) expressing a CACNA1Isv1 polypeptide comprising SEQ ID NO 4 or fragment thereof from a recombinant nucleic acid;
(b) providing to said polypeptide a labeled CACNA1I ligand that binds to said polypeptide and a test preparation comprising one or more compounds; and
(c) measuring the effect of said test preparation on binding of said labeled CACNA1I ligand to said polypeptide, wherein a test preparation that alters the binding of said labeled CACNA1I ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell

16. The method of claim 13, wherein said polypeptide is expressed from an expression vector

17. The method of claim 13, wherein said CACNA1Isv1 ligand is an CACNA1I inhibitor.

18. The method of claim 16, wherein said expression vector comprises SEQ ID NO 3 or a fragment of SEQ ID NO 3.

19. The method of claim 16, wherein said polypeptide comprises SEQ ID NO 4 or a fragment of SEQ ID NO 4.

20. A method of screening for CACNA1Isv1 activity comprising the steps of:
(a) contacting a cell expressing a recombinant nucleic acid encoding CACNA1Isv1 comprising SEQ ID NO 4 with a test preparation comprising one or more test compounds; and
(b) measuring the effect of said test preparation on ion channel activity.
